(19) United States
(12) Patent Application Publication
(10) Pub. No.: US 2009/0054493 A1
(43) Pub. Date: Feb. 26, 2009

(54) THIAZOLE DERIVATIVES AS PPAR DELTA LIGANDS AND THEIR MANUFACTURING PROCESS

(75) Inventors: Heonjoong Kang, Kyeongki-do (KR); Jungyeob Ham, Seoul (KR); Hoosang Hwang, Kyeongki-do (KR)

Correspondence Address: LOWE HAUPTMAN HAM & BERNER, LLP, 1700 DIAGONAL ROAD, SUITE 300, ALEXANDRIA, VA, 22314, US

(73) Assignee: Heonjoong Kang Kang, Seoungnam-city (KR)

(21) Appl. No.: 11/816,925

(22) PCT Filed: Feb. 24, 2006
(86) PCT No.: PCT/KR06/00663

§ 371 Date: May 30, 2008

(30) Foreign Application Priority Data
Feb. 25, 2005 (KR) 10 -2005-0015663
Feb. 24, 2006 (KR) 10-2006-0018360

(51) Int. Cl. A61K 31/426; C07D 277/24; C07D 417/12; A61P 3/04; A61P 3/10; A61K 31/4439

(52) U.S. Cl. 514/342; 548/203; 546/269.7; 514/365

(57) ABSTRACT

The present invention relates to novel thiazole derivative compounds having activity for peroxisome prolif erator-activated receptor δ (PPARδ), as well as their intermediates and synthesis methods thereof.

### TECHNICAL FIELD

The present invention relates to novel thiazole derivatives represented by Formula I, as peroxisome proliferator-activated receptor δ (PPAR δ)-activating ligands, which can be used for the treatment of obesity, hyperlipidemia, arteriosclerosis and diabetes, as well as their intermediates and preparation methods thereof:

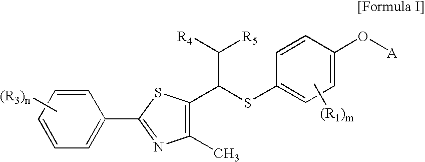

wherein A is hydrogen, R₂ or

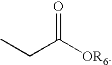

### BACKGROUND ART

Of nuclear receptors, a peroxisome proliferator-activated receptor (PPAR) includes three subtypes: PPARα, PPARγ, PPARδ (Nature, 1990, 347, p 645-650, Proc. Natl. Acad. Sci. USA 1994, 91, p 7335-7359). PPARα, PPARγ and PPARδ have functions distinguished according to in vivo tissues and are expressed in different sites. PPARα is expressed mainly in the human heart, kidneys, skeletal muscle and colon (Mol. Pharmacol. 1998, 53, p 14-22, Toxicol. Lett. 1999, 110, p 119-127, J. Biol. Chem. 1998, 273, p 16710-16714), and is associated with the β-oxidation of peroxisome and mitochondria (Biol. Cell. 1993, 77, p 67-76, J. Biol. Chem. 1997, 272, p 27307-27312). PPARγ is known to be expressed weakly in a skeletal muscle, but expressed largely in fat tissue, and thus involved in the differentiation of fat cells, the storage of energy in the form of fat, and the regulation of homeostasis of insulin and sugar (Moll. Cell. 1999, 4, p 585-594, p 597-609, p 611-617). PPARδ has been evolutionally conserved in Vertebrata, such as mammals, including human beings, rodents and Ascidiacea. Those found so far have been known as PPARβ in Xenopus laevis (Cell 1992, 68, p 879-887), and as NUCI (Mol. Endocrinol. 1992, 6, p 1634-1641), PPARδ (Proc. Natl. Acad. Sci. USA 1994, 91, p 7355-7359), NUCI (Biochem. Biophys. Res. Commun. 1993, 196, p 671-677), FAAR (J. Bio. Chem. 1995, 270, p 2367-2371) and the like, human beings, but these names were recently standardized as PPARδ. In human beings, PPARδ is known to be present in chromosome 6p 21.1-p 21.2, whereas, in rats, the mRNA of PPARδ is found in the cells of various sites, but the amount thereof is shown to be lower than that of PPARα and PPARγ (Endocrinology 1996, 137, p 354-366, J. Bio. Chem. 1995, 270, p 2367-2371, Endocrinology 1996, 137, p 354-366). According to study results so far, PPARδ is known to play an important in an expression process (Genes Dev. 1999, 13, p 1561-1574.), and to perform physiological functions, including the differentiation of nervous cells in the central nervous system (CNS) (J. Chem. Neuroanat 2000, 19, p 225-232), and wound healing by anti-inflammatory effect (Genes Dev. 2001, 15, p 3263-3277, Proc. Natl. Acad. Sci. USA 2003, 100, p 6295-6296). Recent studies demonstrated that PPARδ is associated with the differentiation of fat cells and the metabolism of fat (Proc. Natl. Acad. Sci. USA 2002, 99, p 303-308, Mol. Cell. Biol. 2000, 20, p 5119-5128), and it was found that PPARδ activates the expression of key genes associated with β-oxidation and uncoupling proteins (UCPs) associated with energy metabolism, in a fatty acid degradation process (Nature 2000, 406, p 415-418, Cell 2003, 113, p 159-170, PLoS Biology 2004, 2, p 1532-1539). Furthermore, the activation of PPARδ makes it possible to increase HDL levels and improve type II diabetes without changing bodyweight and (Proc. Natl. Acad. Sci. USA 2001, 98, p 5306-5311, 2003, 100, p 15924-15929) and to suppress arteriosclerosis-associated genes so as to treat arteriosclerosis (Science, 2003, 302, p 453-457). Accordingly, the regulation of fat metabolism by PPARδ provides an important solution required to treat obesity, diabetes, hyperlipidemia and arteriosclerosis.

The crystal structure of Apo-PPARδ LBD was determined on the basis of the already known structure of PPARγ (Nature 1998, 395, p 137-143) and it was reported that the structure of LBD was interestingly similar between PPAR δ and PPARγ, and particularly, the size of ligand-binding pockets was substantially equal between the two PPARs (Mol. Cell. 1999, 3, p 397-403). However, different ligands selective according to the pocket shape will bind to the PPARs, and therefore a difference in function between the PPARs will be shown. As a result of examining the crystal structure of PPARδ LBD in further detail, it consists of 13 α-helixes and 4 small β-strands, and its ligand-binding pocket is Y-shaped and has a size of about 1300 Å³. It can be seen that the entrance of the ligand-binding pocket is about 100 Å² in size, and its periphery consists of polar amino acids. The binding assay of natural eicosapentaenoic acid (EPA) and synthetic ligand GW2433 showed that the Y473 amino acid at the AF-2 site of crystal structure of PPARδ makes a hydrogen bond with the carboxylic acid of the ligand (Proc. Natl. Acad. Sci. USA 2001, 98, p 13919-13924). This is supported by the fact that one side of the structure of most PPARδ-activated ligands consists of a functional group that can make a hydrogen bond. Accordingly, it can be inferred that the binding of a co-activator associated with PPARδ is well kept by the stabilization of the hydrogen bond between the AF-2 helix and the ligand. From the crystal structure of the ligand-binding pocket of PPARδ, it also was found that the active ligand requires a hydrophobic functional group at the other side. As a result, it is inferred that, due to the size of the ligand-binding pocket of PPARδ, various types of ligands can be bound and therefore show a difference in the activation thereof (Nature 1998, 391, p 79-82).

In the case of PPARδ, the development of highly selective synthetic ligands was relatively insufficient compared to PPARα and PPARγ. A selective ligand developed in the first stage is L-631033 reported by the research team of Merk Co. (J. Steroid Biochem. Mol. Biol. 1997, 63, p 1-8), in which the L-631033 ligand was made by introducing a functional group capable of fixing a side chain, based on the structure of natural fatty acids. Also, the same research team reported more effective ligand L-165041 (J. Med. Chem. 1996, 39, p 2629-2654), which is a compound already known as a leukotriene agonist which acts also as an activator on human PPARδ. This substance showed a 10-fold higher selectivity for hPPARδ than PPARα and PPARγ, and had an EC₅₀ value of 530 nM. However, in a test on rodents, it had almost no selectivity for PPARγ. Other ligands L-796449 and L-783483 had a significantly improved affinity (EC₅₀=7.9 nM), but showed no selectivity for other hPPAR subtypes. The research team of Glaxo-Smith-Kline Co. reported PPARα activator GW2433 a Y-shaped ligand having a crystal structure similar to that of the PPARδ ligand pocket (Chem. Biol. 1997, 4, p 909-918). This ligand was reported to be spatially well bound to the ligand-binding pocket, since it has a Y-shaped structure containing a benzene structure, unlike ligands developed so far. However, this ligand is a double-activating ligand showing activity also for hPPARα and showed reduced selectivity for PPARδ. PPARδ-selective ligand GW501516 ([2-methyl-4-[[[4-methyl-2-[4-(trifluoromethyl)phenyl]-1,3-thiazol-5-yl]methyl]sulfanyl]phenoxy]acetic acid) recently developed by Glaxo-Smith-Kline Co. showed excellent physiological activity compared to the earlier developed ligand (Proc. Natl. Acad. Sci. USA 2001, 98, p 5306-5311). The ligand GW501516 had a very good affinity (1-10 nM) for PPARδ and showed a 1000-fold higher selectivity for PPARα and PPARδ. Accordingly, it is thought that, in future experiments associated with PPARδ, an experiment based on GW501516 will be effective. However, PPARδ activities obtained by ligands developed so far are results shown by binding to 30-40% of the total region of the ligand-binding pocket.

### DISCLOSURE OF INVENTION

### Technical Problem

Accordingly, in order to confirm the exact effects of PPARδ on obesity, hyperlipidemia, arteriosclerosis and diabetes, there is a need for the development of novel ligands having a shape similar to a ligand-binding pocket and showing high selectivity and activity, as well as an economically advantageous preparation method thereof.

### Technical Solution

The present invention relates to novel thiazole derivatives represented by Formula I, as peroxisome proliferator-activated Receptor δ (PPARδ)-activating ligands, which can be used for the treatment of obesity, hyperlipidemia, arteriosclerosis and diabetes, as well as their intermediates and preparation methods thereof:

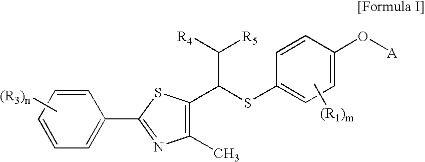

wherein A is hydrogen, R₂ or

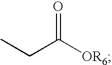

R₁ is a hydrogen atom, a C₁₋₄ alkyl group, a C₁₋₄ alkyloxy group, a C₁₋₄ alkylthioxy group, a C₁₋₄ alkylamine group, a fluorine atom or a chlorine atom;
M is an integer from 0 to 4;
R₂ is a phenol-protecting group selected from among C₁₋₄ lower alkyl groups, allyl groups, alkylsilyl groups, alkylarylsilyl groups and a tetrahydropyranyl group;
R₃ groups are different from each other and denote a hydrogen atom, a halogen atom, or a C₁₋₄ alkyl or alkoxy group substituted or unsubstituted with halogen;
N is an integer from 0 to 5;

### R₄ is

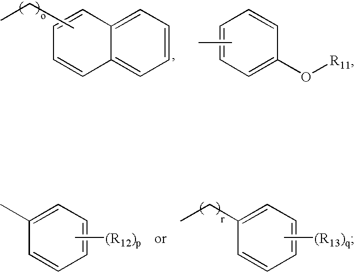

R₅ is a hydrogen atom, a hydroxyl group or a C₁₋₄ alkyl group;
R₆ is a carboxylic acid protecting group having C₁₋₄ alkyl, an allyl group, a hydrogen atom or an alkali metal;
R₁₁ is an arylaminoalkyl group or an alkylaminoalkyl group;
R₁₂ is a halogen atom, a cyano group, or a C₁₋₄ alkyl or alkoxy group substituted or unsubstituted with halogen;
R₁₃ is a hydrogen atom, a halogen atom, a cyano group, a C₁₋₄ alkyl or alkoxy group substituted or unsubstituted with halogen;
o, p and q are each independently an integer from 1 to 5; and r is an integer from 1 to 9.

The thiazole derivative compounds according to the present invention include racemates or optical isomers represented by Formulas VI, VII and IX, and compounds of Formula X, which can be prepared from compounds of Formula IX:

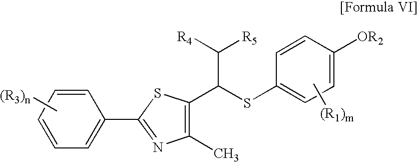

wherein R₁ to R₅, m and n have the same meanings as described in Formula I above;

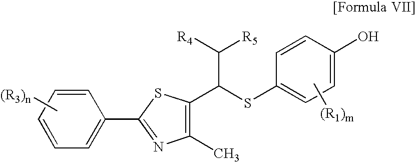

wherein R₁, R₃ to R₅, m and n have the same meanings as described in Formula I above;

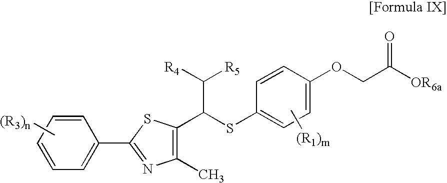

wherein R₁, R₃ to R₅, m and n have the same meanings as described in Formula I above, and R₆ₐ is a carboxylic acid protecting group having a C₁₋₄ alkyl group, or an allyl group;

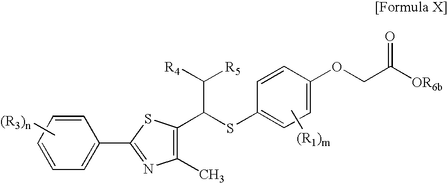

wherein R₁, R₃ to R₅, m and n have the same meanings as described in Formula I above, and R_{6b} is a hydrogen atom or an alkali metal.

The thiazole derivative compounds of Formula X according to the present invention are characterized by having activity for a peroxisome proliferator-activated receptor δ (PPARδ).

The novel compounds according to the present invention can be prepared through the following reaction pathways.

As shown in the following reaction scheme, the phenol group of a 4-halogen phenol compound of Formula II as a starting material is protected with an alkylsilyl group to obtain a compound of Formula III, which is substituted with lithium and allowed to react with sulfur and a compound of Formula IV to obtain a compound of Formula V. The formula V compound is allowed to react various electrophilic compounds in the presence of a strong base to synthesize compounds of Formula VI, followed by removal of the silyl protecting group from the phenol group, thus obtaining compounds of Formula VII. In another method, the phenol group of the formula II compound is protected with a Grignard reagent, and the halogen of the compound is substituted with lithium, and the resulting compound is allowed to react with sulfur and a compound of Formula IV to form thioether. The thioether is allowed to react with a strong base without separation and are then allowed to sequentially react with various electrophilic compounds (O═CR₄-R₅ or X₃—CHR₄R₅), whereby compounds of Formula VII can be obtained in a single process. The formula VII compounds thus obtained are allowed to react with alkyl halogen acetate of Formula VIII in the presence of inorganic salt to synthesize compounds of Formula IX, followed by ester hydrolysis, so as to obtain compounds of Formula X. Based on the finding that the compounds of Formula X can be prepared by the above-described method, the present invention has been completed.

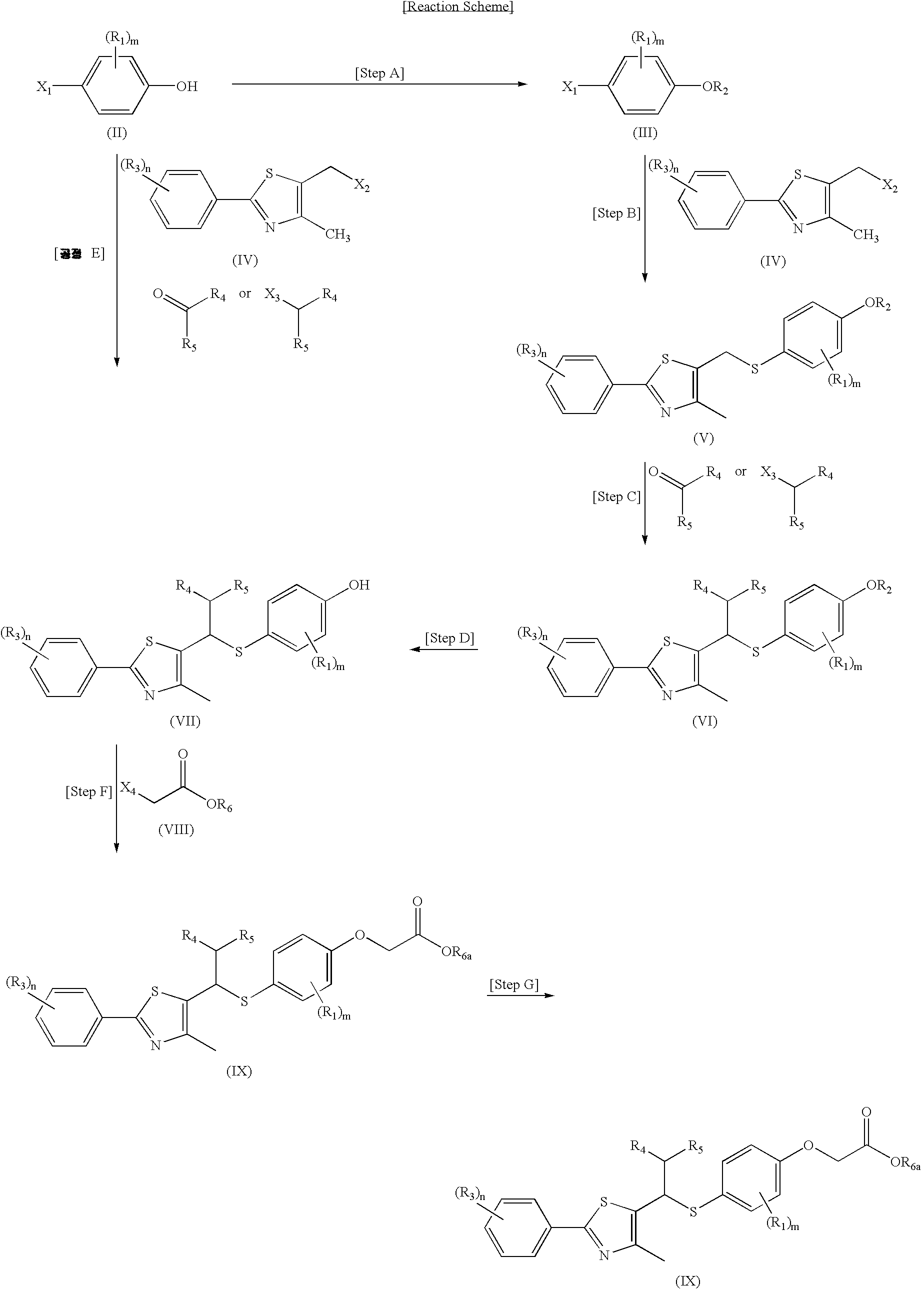

wherein R₁ is a hydrogen atom, a C₁₋₄ alkyl group, a C₁₋₄ alkyloxy group, a C₁₋₄ alkylthioxy group, a C₁₋₄ alkylamine group, a fluorine atom or a chlorine atom;
m is an integer from 0 to 4;
R₂ is a phenol-protecting group selected from among C₁₋₄ lower alkyl groups, allyl groups, alkylsilyl groups, alkylarylsilyl groups and a tetrahydropyranyl group;
R₃ groups are different from each other and denote a hydrogen atom, a halogen atom, or a C₁₋₄ alkyl or alkoxy group substituted or unsubstituted with halogen;
n is an integer from 0 to 5;

### R₄ is

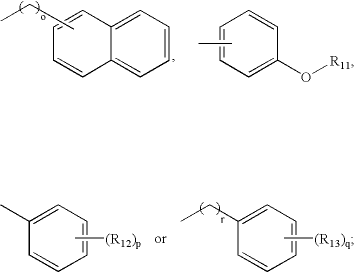

R₅ is a hydrogen atom, a hydroxyl group, or a C₁₋₄ alkyl group;
R₆ is a carboxylic acid protecting group having C₁₋₄ alkyl, allyl group, a hydrogen atom or an alkali metal;
R₁₁ is an arylaminoalkyl group or an alkylaminoalkyl group;
R₁₂ is a halogen atom, a cyano group, or a C₁₋₄ alkyl or alkoxy group substituted or unsubstituted with halogen;
R₁₃ is a hydrogen atom, a halogen atom, a cyano group, or a C₁₋₄ alkyl or alkoxy group substituted or unsubstituted with halogen;
o, p and q are each independently an integer from 1 to 5; and r is an integer from 1 to 9.

Specifically, an object of the present invention is to provide novel PPARδ-activating ligands represented by Formula X, which can be used as agents for treating obesity, hyperlipidemia, arteriosclerosis and diabetes.

Another object of the present invention is to provide a method for preparing compounds of Formula VI, which comprises reacting a compound of Formula II with a phenol-protecting group to obtain a compound of Formula III, subjecting the formula III compound to halogen-lithium substitution, reacting the resulting compound with sulfur (S) and a compound of Formula IV without separation and purification so as to prepare a compound of Formula V, reacting the formula V compound with a strong base and then with various electrophilic compounds.

Still another object of the present invention is to provide a method for preparing compounds of Formula VII by removing the phenol-protecting group from the compounds of Formula VI.

Still another object of the present invention is to provide a method for preparing compounds of Formula VII through a single process in a convenient manner, the method comprising protecting the phenol group of a phenolic compound of Formula II with a Grignard reagent without conducting a special reaction for introducing a protecting group, subjecting the protected compound to halogen-to-lithium substitution, reacting the resulting compound with sulfur (S) and then with a compound of Formula IV to prepare a thioether compound, and reacting the thioether compound with a strong base and electrophilic compounds.

Still another object of the present invention is to provide a method for preparing compounds of Formula IX, comprising reacting the compounds of Formula VII with alkyl halogen acetate and inorganic salt.

Still another object of the present invention is to provide a method for preparing compounds of Formula X by hydrolyzing the ester compounds of Formula IX.

Among the compounds represented by Formula X, the following compounds are novel compounds, and intermediates of Formulas V, VI, VII and IX for preparing these compounds are also novel compounds: 2-[4-[1-[2-[4-(trifluoromethyl)phenyl]4-methylthiazol-5-yl]-3-phenylpropylthio]-2-methylphenoxy]acetic acid, 2-[4-[1-[2-[4-(trifluoromethyl)phenyl]-4-methylthiazol-5-yl]-4-phenylbutylthio]-2-methylphenoxy]acetic acid, 2-[4-[1-[2-[4-(trifluoromethyl)phenyl]-4-methylthiazol-5-yl]-5-phenylpentylthio]-2-methylphenoxy]acetic acid, 2-[4-[1-[2-[4-(trifluoromethyl)phenyl]-4-methylthiazol-5-yl]-6-phenylhexylthio]-2-methylphenoxy]acetic acid, 2-[4-[1-[2-[4-(trifluoromethyl)phenyl]-4-methylthiazol-5-yl]-8-phenyloctylthio]-2-methylphenoxy]acetic acid, 2-[4-[1-[2-[4-(trifluoromethyl)phenyl]-4-methylthiazol-5-yl]-11-phenylundecylthio]-2-methylphenoxy]acetic acid, 2-[4-[2-(2-chloro-6-fluorophenyl)-1-[2-[4-(trifluoromethyl)phenyl]-4-methylthiazol-5-yl]ethylthio]-2-methylphenoxy]acetic acid, 2-[4-[2-(4-cyanophenyl)-1-[2-[4-(trifluoromethyl)phenyl]-4-methylthiazol-5-yl]ethylthio]-2-methylphenoxy]acetic acid, 2-[4-[1-[2-[4-(trifluoromethyl)phenyl]-4-methylthiazol-5-yl]-2-(naphthalene-3-yl)ethylthio]-2-methylphenoxy]acetic acid, 2-[4-[2-[4-(trifluoromethyl)phenyl]-1-[2-[4-(trifluoromethyl)phenyl]-4-methylthiazol-5-yl]ethylthio]-2-methylphenoxy]acetic acid, 2-[4-[1-[2-[4-(trifluoromethyl)phenyl]-4-methylthiazol-5-yl]-2-(3,5-dimethoxyphenyl)ethylthio]-2-methylphenoxy]acetic acid, 2-[4-[1-[2-[4-(trifluoromethyl)phenyl]-4-methylthiazol-5-yl]-2-(perfluorophenyl)ethylthio]-2-methylphenoxy]acetic acid, 2-[4-[2-(4-bromophenyl)-1-[2-[4-(trifluoromethyl)phenyl]-4-methylthiazol-5-yl]ethylthio]-2-methylphenoxy]acetic acid, 2-[4-[2-[2-fluoro-6-(trifluoromethyl)phenyl]-1-[2-[4-(trifluoromethyl)phenyl]-4-methylthiazol-5-yl]ethylthio]-2-methylphenoxy]acetic acid, 2-[4-[1-[2-[4-(trifluoromethyl)phenyl]-4-methylthiazol-5-yl]-2-(2,6-difluorophenyl)ethylthio]-2-methylphenoxy]acetic acid, 2-[4-[2-(2,6-dichlorophenyl)-1-[2-[4-(trifluoromethyl)phenyl]-4-methylthiazol-5-yl]ethylthio]-2-methylphenoxy]acetic acid, 2-[4-[1-[2-[4-(trifluoromethyl)phenyl]-4-methylthiazol-5-yl]-2-(2,4-difluorophenyl)ethylthio]-2-methylphenoxy]acetic acid, 2-[4-[1-[2-[4-(trifluoromethyl)phenyl]-4-methylthiazol-5-yl]-2-(2,3,4-trifluorophenyl)ethylthio]-2-methylphenoxy]acetic acid, 2-[4-[2-(2-chloro-5-fluorophenyl)-1-[2-[4-(trifluoromethyl)phenyl]-4-methylthiazol-5-yl]ethylthio]-2-methylphenoxy]acetic acid, 4-[3-(2-chloro-6-fluorophenyl)-2-hydroxy-1-[4-methyl-2-(4-trifluoromethyl-phenyl)-thiazol-5-yl]propylsulfanyl]-2-methyl-phenoxy]acetic acid, 4-[2-hydroxy-1-[4-methyl-2-(4-trifluoromethyl-phenyl)thiazol-5-yl]-[1-phenyl-undecylsulfanyl]-2-methyl-phenoxy]acetic acid, 4-[2-hydroxy-1-[4-methyl-2-(4-trifluoromethyl-phenyl)-thiazol-5-yl]-2-phenyl-ethylsulfanyl]-2-methyl-phenoxy]acetic acid, 2-[4-[2-(2-chloro-6-fluorophenyl)-1-[2-[3-fluoro-4-(trifluoromethyl)phenyl]-4-methylthiazol-5-yl]ethylthio]-2-methylphenoxy]acetic acid, 2-[4-[1-[2-[3-fluoro-4-(trifluoromethyl)phenyl]-4-methylthiazol-5-yl]-2-(3,4,5-trifluorophenyl)ethylthio]-2-methylphenoxy]acetic acid, 2-[4-[1-[2-[3-fluoro-4-(trifluoromethyl)phenyl]-4-methylthiazol-5-yl]-2-(2-fluoro-6-(trifluoromethyl)phenyl)ethylthio]-2-methylphenoxy]acetic acid, 2-[4-[1-[2-[3-fluoro-4-(trifluoromethyl)phenyl]-4-methylthiazol-5-yl]-2-(2,6-difluorophenyl)ethylthio]-2-methylphenoxy]acetic acid, 2-[4-[2-(2,6-dichlorophenyl)-1-[2-[3-fluoro-4-(trifluoromethyl)phenyl]-4-methylthiazol-5-yl]ethylthio]-2-methylphenoxy]acetic acid, 2-[4-[1-[2-[3-fluoro-4-(trifluoromethyl)phenyl]-4-methylthiazol-5-yl]-2-(2,4-difluorophenyl)ethylthio]-2-methylphenoxy]acetic acid, 2-[4-[2-(2-chloro-5-fluorophenyl)-1-[2-[3-fluoro-4-(trifluoromethyl)phenyl]-4-methylthiazol-5-yl]ethylthio]-2-methylphenoxy]acetic acid, and potassium 2-[4-[2-(2-chloro-6-fluorophenyl)-1-[2-[4-(trifluoromethyl)phenyl]-4-methylthiazol-5-yl]ethylthio]-2-methylphenoxy]acetate.

Accordingly, the present invention provides novel useful compounds.

In the inventive compounds, R₁ denotes a hydrogen atom, a C₁₋₄ alkyl group, a C₁₋₄ alkyloxy group, a C₁₋₄ alkylthioxy group, a C₁₋₄ alkylamine group, a fluorine atom or a chlorine atom. Each of R₁ groups is at an ortho or meta position with respect to the phenol group, and the number (m) of R₁ groups is 0-4.

R₂ is a phenol-protecting group, such as C₁₋₄ lower alkyl, allyl, alkylsilyl or alkylarylsilyl such as trimethylsilyl, tert-butyldiphenylsilyl, triisopropylsilyl or tert-butyldimethylsilyl, or tetrahydropyranyl. Among these protecting groups, preferred is tert-butyl, tetrahydropyranyl, or a silylated protecting group.

R₃ groups are different from each other and denote a hydrogen atom, a halogen atom or a C₁₋₄ alkyl or alkoxy group substituted or unsubstituted with halogen, and the number (n) of R₃ groups is 0-5.

R₄ denotes

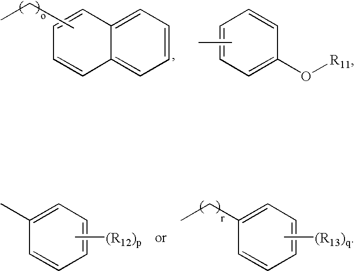

R₅ denotes a hydrogen atom, a hydroxyl group or a C₁₋₄ alkyl group.

R₆ is a carboxylic acid protecting group having a C₁₋₄ alkyl group (e.g., methyl, ethyl, n-propyl, iso-propyl, n-butyl, sec-butyl or tert-butyl), an allyl, a hydrogen atom or an alkali metal (Li⁺, Na⁺, K⁺).

R₁₁ is an arylaminoalkyl group, such as methyl pridinyl amino ethyl, methyl phenyl amino ethyl, or t-butyl phenyl amino ethyl, or an alkylaminoalkyl group, such as methyl amino ethyl, t-butyl amino ethyl or ethyl amino propyl.

R₁₂ is a halogen atom, a cyano group or a C₁₋₄ alkyl or alkoxy group substituted or unsubstituted with halogen.

R₁₃ is a hydrogen atom, a halogen atom, a cyano group or a C₁₋₄ alkyl or alkoxy group substituted or unsubstituted with halogen.

o, p and q are each independently an integer from 1 to 5.

r is an integer from 1 to 9.

X₁ is a halogen atom, such as a bromine atom (Br) and a iodine atom (I).

X₂ denotes a leaving group in nucleophilic reaction. As the leaving group, conventional leaving groups can be used, for example, halogen atoms, such as chlorine, bromine or iodine, methanesulfonyloxy (MsO⁻) and p-toluenesulfonyloxy (TsO⁻). Among these leaving groups, preferred are chlorine and bromine.

X₃ denotes a leaving group. As the leaving group, conventional leaving groups, for example, halogens, methanesulfonyloxy (MsO⁻) and p-toluenesulfonyloxy (TsO⁻), can be used. The halogens include fluorine, chlorine, bromine and iodine. Among these leaving groups, preferred are halogens, and more preferred are chlorine, bromine and iodine.

X₄ denotes a halogen atom, such as chlorine (Cl), bromine (Br) or iodine (I).

The compounds of Formulas (I) and (II) and the electrophilic compounds, used as raw materials or intermediates in the preparation method according to the present invention, are known compounds which can be commercially easily available or easily prepared according to the literature.

Hereinafter, the present invention will be described in further detail.

Step A: Preparation of Compound Represented by Formula III

In order to obtain a compound represented by Formula III, a compound represented by Formula II is preferably allowed to react with a compound conventionally used as a phenol protecting group, in the presence of a base.

Non-protonic polar solvents which can be used in this step may include N,N-dimethylformamide, N,N-dimethylacetamide, dimethylsulfoxide, acetonitrile, acetone, ethyl acetate, carbon tetrachloride, chloroform and dichloromethane. Ether solvents which can be used in this step may include tetrahydrofuran, dioxane, dimethoxyethane, diethyleneglycoldimethylether and triethyleneglycoldimethylether. Aromatic hydrocarbons may include benzene, toluene and xylene. Among these solvents, preferred are non-protonic polar solvents, and more preferred are N,N-dimethylformamide, chloroform and dichloromethane.

Bases which can be used in this step include amine bases, such as pyridine, triethylamine, imidazole and N,N-dimethylaminopyridine, and if alkyl or allylether is used as the protecting group, sodium hydroxide, potassium hydroxide, sodium carbonate or potassium carbonate will be used as the base. Among these bases, preferred bases are imidazole and potassium carbonate.

The tetrahydropyranyl-protecting group is obtained by reacting 3,4-dihydro-2H-pyran with alkyl or allyltriphenylphosphonium bromide in the presence of a catalyst.

The reaction temperature may vary depending on the kind of solvent used, but is generally −10 to 80° C., and preferably 0 to room temperature (25° C.). The reaction time may vary depending on the reaction temperature and the kind of solvent used, but is generally 1 hour to 1 day, and preferably 4 hours or shorter.

Step B: Preparation of Compound Represented by Formula V

A compound represented by Formula V is obtained in a single process by subjecting the compound of Formula III to halogen-to-lithium substitution, sulfur introduction and then reaction with a compound of Formula IV.

Anhydrous solvents which can be used in this step include diethylether, tetrahydrofuran, hexane, heptane and a mixture of two or more thereof. Among these solvents, the most preferred solvents are diethylether, tetrahydrofuran and a mixed solvent of diethylether and tetrahydrofuran.

Metal reagents which can be used in the halogen-to-metal substitution reaction include metals, such as lithium metal and magnesium metal, and organic metal reagents, such as n-butyllithium, sec-butyllithium and tert-butyllithium. Among these reagents, preferred are the organic metal reagents, and more preferred are n-butyllithium and tert-butyllithium.

The reaction temperature may vary depending on the kind of solvent used, but is generally −100 to 25° C., and preferably −75° C. to room temperature for the halogen-to-lithium substitution and the sulfur introduction reaction, and room temperature (25° C.) for the reaction with the compound of Formula III. The reaction time may vary depending on the reaction temperature and the kind of solvent used, but is generally 30 minutes to 4 hours, and preferably 1 hour or shorter.

Step C: Preparation of Compound Represented by Formula VI

A compound represented by Formula VI is obtained by treating the α-proton of thioether of the compound of Formula V with a strong base to prepare a nucleophile which is then allowed to react with various electrophilic compounds.

Anhydrous solvents which can be used in this step include diethylether, tetrahydrofuran, hexane, heptane, and a mixture of two or more thereof. Among these solvents, preferred solvents are diethylether, tetrahydrofuran and a mixed solvent of diethylether and tetrahydrofuran.

Strong base reagents which can be used in the alpha-hydrogen extraction reaction include potassium tert-butoxide (t-BuOK), lithium diisopropylamide (LDA), n-butyllithium, sec-butyllithium and tert-butyllithium. Among these reagents, the most preferred is tert-butyllithium.

The electrophilic compounds that react with the nucleophilic thioether compound are known compounds which can be commercially easily available or easily prepared according to the literature and contain halogen, aldehyde or ketone. These compounds are added for reaction after dissolution in anhydrous solvent or without dissolution.

The reaction temperature may vary depending on the kind of solvent used, but is generally −78 to 25° C. Preferably the alpha-hydrogen extraction with the strong base is conducted at −75° C., and the electrophilic compounds are added at −75° C. and reacted while elevating the temperature slowly to room temperature (25° C.). The reaction time may vary depending on the reaction step, but is 10-30 minutes for the alpha-hydrogen extraction with the strong base and 30-90 minutes for the reaction with the electroophilic compounds.

Step D: Preparation of Compound Represented by Formula VII

A compound of Formula VII is obtained by removing the phenol-protecting group from the compound of Formula VI.

Polar solvents which can be used in this step include N,N-dimethylformamide, N,N-dimethylacetamide, dimethylsulfoxide, acetonitrile, acetone, ethylacetate, carbon tetrachloride, chloroform and dichloromethane. Ether solvents may include tetrahydrofuran, dioxane, dimethoxyethane and diethyleneglycoldimethylether. Alcohol solvents may include methanol and ethanol. Aromatic hydrocarbons may include benzene, toluene and xylene. Among these solvents, preferred are the polar solvents, and the most preferred is tetrahydrofuran.

For removal of the phenol-protecting group, Lewis acids, such as trimethylsilyl iodide, sodium ethane thioalcohol, lithium iodide, aluminum halide, boron halide and trifluoroacetic acid are used to remove protecting groups, such as methyl, ethyl, tert-butyl, benzyl and allylether. Also, fluorides, such as tetrabutylammonium fluoride (Bu₄N⁺F⁻), halogenic acids (fluoric acid, hydrochloric acid, bromic acid and iodic acid), potassium fluoride are used to remove silylated protecting groups, such as trimethylsilyl, tert-butyldiphenylsilyl, triisopropylsilyl and tert-butyldimethylsilyl. Among these groups for removal for the silylated protecting groups, preferred are fluorides and more preferred is tetrabutylammonium fluoride.

The reaction temperature may vary depending on the kinds of deprotecting group and solvent used, but is generally 0-120° C. and preferably 10-25° C. The reaction time may vary depending on the reaction time, but is generally 30 minutes to 1 day, and preferably 2 hours or shorter.

Step E: Preparation of Compound Represented by Formula VII

To obtain a compound represented by Formula VII, the phenol group of the compound represented by Formula II is protected with a Grignard reagent, and the protected compound is allowed to react with an organic metal reagent, sulfur (S) and then a compound of Formula IV. The resulting compound is then allowed to react with electrophilic compounds in the presence of a strong base. This step E suggests a very convenient method for carrying out the reaction in a single process.

Hereinafter, sub-steps of step E will be described.

Protection of Phenol Group with Grignard Reagent (Step E-1)

Anhydrous solvents which can be used in this step include diethylether, tetrahydrofuran, hexane, heptane and a mixed solvent of two or more thereof. Among these solvents, preferred is diethylether, tetrahydrofuran or a mixed solvent of diethylether and tetrahydrofuran.

A Grignard reagent used is methyl, ethyl, n-propyl, iso-propyl, n-butyl, sec-butyl magnesium chloride (R₂MgCl) or alkyl magnesium bromide (R₂MgBr). Among these reagents, the most preferred is iso-propyl magnesium chloride (CH₃)₂CHMgCl).

The reaction temperature may vary depending on the kind of solvent used, but is generally −20 to 40° C., and preferably 0° C. to room temperature (25° C.). The reaction time may vary depending on the reaction temperature and the kind of solvent used, but is generally 10-60 minutes, and preferably 10-30 minutes.

Halogen-to-Lithium Substitution and Sulfur (S) Introduction (Steps E-2 and E-3)

Organic metal reagents which can be used in halogen-to-lithium substitution reaction include n-butyllithium, sec-butyllithium and tert-butyllithium. Among these metal reagents, preferred is tert-butyllithium.

Sulfur (S) is preferably in the form of fine particle powder and is added for reaction after dissolution in an anhydrous tetrahydrofuran solvent or without dissolution.

The reaction temperature may vary depending on the kind of solvent used, but is generally −78 to 25° C., and preferably −75° C. for the halogen-to-metal substitution reaction, and room temperature (25° C.) starting from −75° C. for the sulfur introduction reaction. The reaction temperature is 10-30 minutes for the halogen-to-metal substitution reaction and 30-90 minutes for the sulfur introduction reaction.

Addition Reaction of Compound Represented by Formula IV [Step E-4]

5-halogenmethyl-4-methyl-2-[4-(trifluoromethyl)phenyl]thiazol of Formula IV used in this step is synthesized according to a known method (WO 2003/106442). Examples of halogen in the formula IV compound include chlorine, bromine and iodine. Among these halogens, preferred is chlorine.

The reaction temperature may vary depending on the kind of solvent used, but is generally −78° C. to 25° C., and preferably 0° C. to 10° C. The reaction time is generally 10-120 minutes, and preferably 10-60 minutes.

Reactions with Various Electrophilic Compounds (Step E-5)

Strong bases which can be used to treat the α-proton of thioether to prepare nucleophilic compounds include potassium tert-butoxide (t-BuO⁻K⁺), lithium diisopropyl amide (LDA), n-butyllithium, sec-butyllithium and tert-butyllithium. Among these bases, tert-butyllithium is most preferable.

The electrophilic compound that reacts with nucleophilic thioether compounds is a known compound which is commercially easily available or easily prepared according to the literature and contains highly reactive halogen, aldehyde or ketone. This compound is added for reaction after dissolution in anhydrous solvent or without dissolution.

The reaction temperature may vary depending on the kind of solvent used, but is generally −78 to 25° C. Preferably, the alpha-hydrogen extraction with the strong base is carried out at −75° C., and the electrophilic compounds are added at −75° C. and allowed to react while elevating the temperature to room temperature (25° C.). The reaction time varies depending on the reaction step, but is 10-30 minutes for the alpha-hydrogen extraction with the strong base, and 30-90 minutes for the reaction with the electrophilic compounds.

Step F: Preparation of Compound Represented by Formula IX

To obtain a compound represented by Formula IX, a compound represented by Formula VII is preferably allowed to react with halogen acetic acid alkyl ester in the presence of a base.

The halogen acetic acid alkyl ester is a known compound which is commercially easily available and in which the halogen is chlorine, bromine or iodine. The most preferred example of the halogen acetic acid alkyl ester is bromoacetic acid methyl ester or bromoacetic acid ethyl ester.

Solvents which can be used in this step include water-soluble solvents, such as N,N-dimethylformamide, N,N-dimethylacetamide, dimethylsulfoxide, acetonitrile, acetone, ethanol and methanol, or a mixture of any one thereof with 1-10% water. Among these solvents, the most preferred is a mixture of acetone or dimethylsulfoxide with 1-5% water.

The base used is not specifically limited regardless of a strong base or weak base as long as it does not adversely affects the reaction, and examples thereof include alkali metal hydrides, such as sodium hydride and lithium hydride, alkaline earth metal hydride such as potassium hydride, alkali metal hydroxides, such as sodium hydroxide and potassium hydroxide, and alkali metal carbonates, such as lithium carbonate, potassium carbonate, potassium hydrogen carbonate and cesium carbonate. Among these bases, preferred is alkali metal carbonate, and more preferred is potassium carbonate.

The reaction temperature is not specifically limited as long as it is below the boiling point of a solvent used, but a reaction at a relatively high temperature is preferably avoided in order to suppress side reactions. The reaction temperature is generally 0-60° C. The reaction temperature may vary depending on the reaction temperature, but is generally 30 minutes to 1 day, and preferably 30-90 minutes.

Step G-1: Preparation of Compound Represented by Formula X

A compound represented by Formula X is prepared by hydrolyzing the carboxylic ester of the compound of Formula IX with a water-soluble inorganic salt in alcohol solvent.

Solvents which can be used in this step include water-soluble alcohol solvents, such as methanol and ethanol.

Bases which can be used in this step include about 0.1-3 N aqueous solutions prepared using alkali metal hydroxides, such as lithium hydroxide, sodium hydroxide and potassium hydroxide, according to the form of carboxylic acid alkali salts. As an acid used to obtain the compound of Formula X in the form of carboxylic acid, acetic acid or 0.1-3N hydrochloric acid aqueous solution is preferably used.

The reaction is preferably carried out at a relatively low temperature in order to inhibit side reactions, and is generally at 0° C. to room temperature. The reaction time may vary depending on the reaction temperature, but is generally 10 minutes to 3 hours, and preferably 30 minutes to 1 hour.

Sep G-2: Preparation of Compound Represented by Formula X

A compound represented by Formula X is prepared by substituting the allyl ester of the compound of Formula IX with a metal salt of 2-ethylhexanoate in an organic solvent in the presence of a metal catalyst.

The solvent used in this step is an anhydrous organic solvents, such as chloroform, dichloromethane or ethyl acetate.

As the metal catalyst, palladium tetrakistriphenylphosphin is preferably used in an amount of 0.01-0.1 equivalent.

The reaction is preferably carried out at a relatively low temperature in order to inhibit side reactions, and is generally conducted at 0° C. to room temperature. The reaction time may vary depending on the reaction temperature, but is generally 10 minutes to 3 hours, and preferably 30 minutes to 1 hour.

This salt compound is separated with high purity by centrifugation. The obtained metal salt-type compound of Formula X is easier to separate than the salt-type compound prepared using the step G-1 (hydrolysis step).

The Y-shaped thiazole compounds of Formula X thus obtained are important substances as ligands for PPARδ. Also, these compounds have chiral carbon, and so stereoisomers thereof exist. Among the compounds of Formula X, R-form or S-form isomers are confirmed to be effective compared to racemates, and the scope of the present invention encompasses the compounds of Formula X, and their stereoisomers, solvates and salts.

### ADVANTAGEOUS EFFECTS

As described above, the novel thiazole derivative compounds according to the present invention have the characteristics of PPARδ-activating ligands and show a high possibility to be used as agents for treating cardiovascular disease, lowering cholesterol levels and treating diabetes and obesity. Also, the inventive preparation method is useful for the preparation of the thiazole derivative compounds.

### BEST MODE

### Mode for Invention

### Examples

Hereinafter, the method according to the present invention will be described in further detail by examples. It will however be obvious to a person skilled in the art that the present invention is not limited to or by these examples.

### Example 1

### Preparation of 4-iodo-2-methyl-phenoxy-tert-butyldimethyl silane (III) [Step A]

3.0 g (12.8 mmol) of 4-iodo-2-methylphenol and 1.74 g (25.6 mmol, 2.0 equivalents) of imidazole were completely dissolved in 45 ml of dimethylformamide. To the solution, 2.12 g (14.1 mmol, 1.1 equivalents) of tert-butyldimethylsilyl chloride was slowly added, and the mixture was stirred at room temperature for 4 hours. After completion of the reaction, the reaction product was extracted with aqueous ammonium chloride solution and ethyl acetate, and the organic layer was dried over magnesium sulfate. The residue was purified with a silica gel column, and the solvent was removed by distillation under reduced pressure, thus obtaining 4.4 g (98% yield) of the title compound.

¹H NMR (300 MHz, CDCl₃) δ7.47 (d, 1H, J=0.6 Hz), 7.35 (dd, 1H, J=8.4, 2.3 Hz), 6.54 (d, 1H, J=8.4 Hz), 2.18 (s, 3H), 1.03 (s, 9H), 0.22 (s, 6H).

¹³C NMR (75.5 MHz, CDCl₃) δ154.3, 139.9, 135.9, 132.3, 121.1, 83.9, 26.2, 18.7, 17.0, −3.8.

### Example 2

### Preparation of 4-bromo-phenoxy-tert-butyldimethyl silane (III) [Step A]

500 mg (2.90 mmol) of 4-bromophenol and 409 mg (6.0 mmol, 2.00 equivalents) of imidazole were completely dissolved in dimethylformamide. To the solution, 436 mg (2.90 mmol, 1.0 equivalent) of tert-butyldimethylsilyl chloride was slowly added, and the mixture was stirred at room temperature for 4 hours. After completion of the reaction, the reaction product was extracted with aqueous ammonium chloride solution and ethyl acetate, and the organic layer was dried over magnesium sulfate. The residue was purified with a silica gel column, and the solvent was removed by distillation under reduced pressure, thus obtaining 811 mg (97% yield) of the title compound.

¹H NMR (300 MHz, CDCl₃) δ7.32 (d, 2H, J=8.8 Hz), 6.72 (d, 2H, J=10.0 Hz), 0.98 (s, 9H), 0.18 (s, 6H)

¹³C NMR (75.5 MHz, CDCl₃) δ155.3, 132.7, 122.3, 114.0, 26.0, 18.6, −4.1

### Example 3

### Preparation of 5-[4-(tert-butyldimethylsilyloxy)-3-methyl-phenylsulfanylmethyl]-4-methyl-2-[(4-trifluoromethyl)phenyl]-thiazole (V) [Step B]

In a nitrogen atmosphere, 1.5 g (4.32 mmol) of 4-iodo-2-methyl-phenoxy-tert-butyldimethyl silane prepared in Example 1 was dissolved in 120 ml of anhydrous tetrahydrofuran and cooled to −78° C. To the solution, 2.54 ml (1.0 equivalent) of tert-butyllithium (1.7 M-hexane solution) was slowly added. The mixture was stirred for 10 minutes, to which 138 mg (4.32 mmol, 1.0 equivalent) of solid phase sulfur was then added at a time at the same temperature. The mixture was allowed to react for 40 minutes until it reached a temperature of 15° C., to which 1.26 g (4.32 mmol, 1.0 equivalent) of 5-chloromethyl-4-methyl-2-[(4-trifluoromethyl)phenyl]-thiazol of Formula III dissolved in 10 ml of anhydrous THF was then slowly added. After reaction for an additional time of about one hour, the reaction was terminated with aqueous ammonium chloride solution, and the reaction product was extracted with ethyl acetate and aqueous salt solution, and the organic layer was dried over magnesium sulfate. After filtration, the solvent was removed by distillation under reduced pressure, and the residue was purified by silica gel column chromatography, thus obtaining 1.85 g (84% yield) of the title compound.

¹H NMR (300 MHz, CDCl₃) δ7.97 (d, 2H, J=8.0 Hz), 7.65 (d, 2H, J=8.2 Hz), 7.17 (d, 1H, J=1.8 Hz), 7.07 (dd, 1H, J=8.2, 2.3 Hz), 6.67 (d, 1H, J=8.3 Hz), 4.10 (s, 2H), 2.20 (s, 3H), 2.15 (s, 3H), 1.00 (s, 9H), 0.20 (s, 6H).

¹³C NMR (75.5 MHz, CDCl₃) δ163.4, 154.9, 151.8, 136.8, 132.6, 130.4, 129.6 (q, J=32 Hz), 126.8, 126.2 (m), 125.2, 119.6, 33.0, 26.1, 18.7, 17.1, 15.2, −3.9.

### Example 4

### Preparation of 5-[1-[3-methyl-4-(tert-butyldimethylsilyloxy)phenylthio]-3-phenylpropyl]-2-[4-(trifluoromethyl)phenyl]-4-methylthiazole (VI) [Step C]

In a nitrogen atmosphere, 510 mg (1.0 mmol) of 5-[4-(tert-butyldimethylsilanyloxy)-3-methyl-phenylsulfanylmethyl]-4-methyl-2-[(4-trifluoromethyl)phenyl]-thiazole prepared in Example 3 was dissolved in 20 ml of anhydrous tetrahydrofuran. The reaction solution was sufficiently cooled to −78° C., to which 1.2 ml (2.0 equivalents) of tert-butyllithium (1.7 M-heptane solution) was then slowly added. While the reaction solution maintained a deep blue color, 137 μl (1.0 mmol) of (2-bromoethyl)benzene was added thereto, and the reaction temperature was slowly elevated to room temperature. After reduction for an additional time of about 30 minutes, the reaction was terminated with aqueous ammonium chloride solution, and the reaction product was extracted with ethyl acetate and aqueous salt solution, and the organic layer was dried over magnesium sulfate. After filtration, the solvent was removed by distillation under reduced pressure, and the residue was purified by silica gel column chromatography, thus obtaining 388 mg (63% yield) of the title compound.

¹H NMR (300 MHz, CDCl₃) δ7.99 (d, 2H, J=8.5 Hz), 7.67 (d, 2H, J=8.2 Hz), 7.19 (m, 5H), 7.04 (d, 1H, J=2.0 Hz), 6.96 (dd, 1H, J=8.3, 2.4 Hz), 6.59 (d, 1H, J=8.3 Hz), 4.19 (dd, 1H, J=8.9, 6.0 Hz), 2.74 (m, 2H), 2.37 (m, 1H), 2.37 (m, 1H), 2.19 (m, 1H), 2.08 (s, 3H), 1.96 (s, 3H), 0.98 (s, 9H), 0.17 (s, 6H).

### Examples 5 to 34

Compounds shown in Table 1 below were prepared in the same manner as described in Example 4, and the NMR data of the prepared compounds are shown in Table 2 below.

TABLE 1

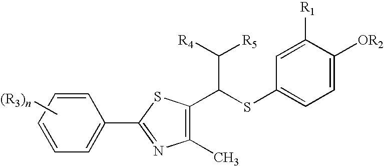

| | R₁ | R₂ | 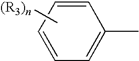 | n | R₄ | R₅ |
|---|---|---|---|---|---|---|
| Example 4 | —CH₃ | —TBDMS | 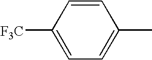 | 1 | 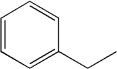 | H |
| Example 5 | —CH₃ | —TBDMS | 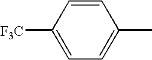 | 1 | 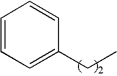 | H |
| Example 6 | —CH₃ | —TBDMS | 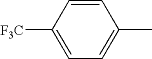 | 1 | 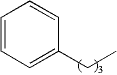 | H |
| Example 7 | —CH₃ | —TBDMS | 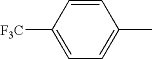 | 1 | 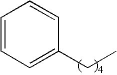 | H |
| Example 8 | —CH₃ | —TBDMS | 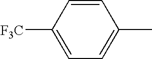 | 1 | 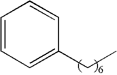 | H |
| Example 9 | —CH₃ | —TBDMS | 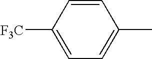 | 1 | 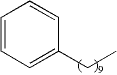 | H |
| Example 10 | —CH₃ | —TBDMS | 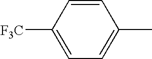 | 1 | 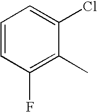 | H |
| Example 11 | —CH₃ | —TBDMS | 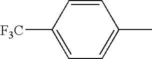 | 1 | 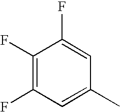 | H |
| Example 12 | —CH₃ | —TBDMS | 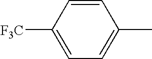 | 1 | 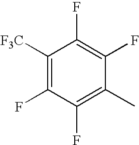 | H |
| Example 13 | —CH₃ | —TBDMS | 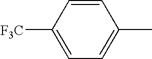 | 1 | 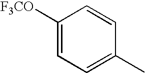 | H |
| Example 14 | —CH₃ | —TBDMS | 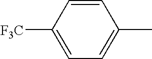 | 1 | 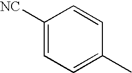 | H |
| Example 15 | —CH₃ | —TBDMS | 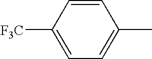 | 1 | 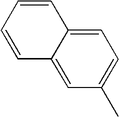 | H |
| Example 16 | —CH₃ | —TBDMS | 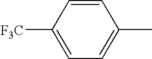 | 1 | 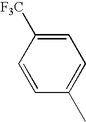 | H |
| Example 17 | —CH₃ | —TBDMS | 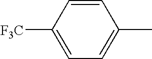 | 1 | 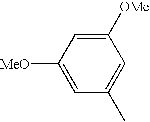 | H |
| Example 18 | —CH₃ | —TBDMS | 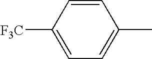 | 1 | 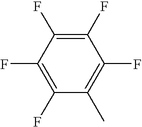 | H |
| Example 19 | —CH₃ | —TBDMS | 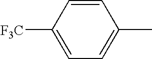 | 1 | 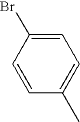 | H |
| Example 20 | —CH₃ | —TBDMS | 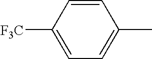 | 1 | 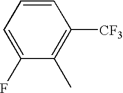 | H |
| Example 21 | —CH₃ | —TBDMS | 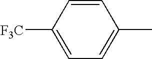 | 1 | 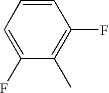 | H |
| Example 22 | —CH₃ | —TBDMS | 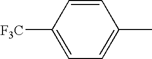 | 1 | 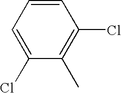 | H |
| Example 23 | —CH₃ | —TBDMS | 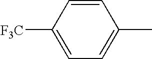 | 1 | 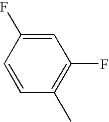 | H |
| Example 24 | —CH₃ | —TBDMS | 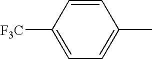 | 1 | 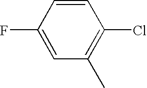 | H |
| Example 25 | —CH₃ | —TBDMS | 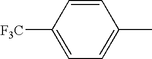 | 1 | OH | 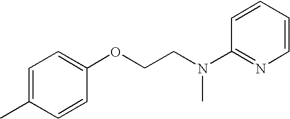 |
| Example 26 | —CH₃ | —TBDMS | 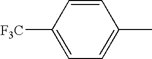 | 1 | OH | 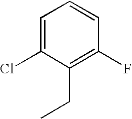 |
| Example 27 | —CH₃ | —TBDMS | 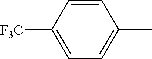 | 1 | OH | 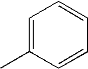 |
| Example 28 | —CH₃ | —TBDMS | 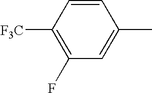 | 2 | 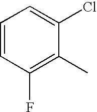 | H |
| Example 29 | —CH₃ | —TBDMS | 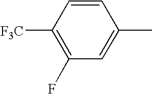 | 2 | 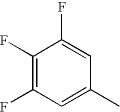 | H |
| Example 30 | —CH₃ | —TBDMS | 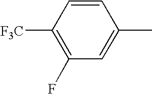 | 2 | 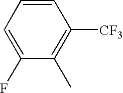 | H |
| Example 31 | —CH₃ | —TBDMS | 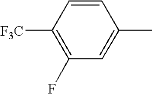 | 2 | 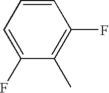 | H |
| Example 32 | —CH₃ | —TBDMS | 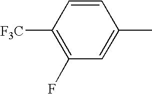 | 2 | 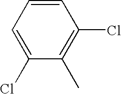 | H |
| Example 33 | —CH₃ | —TBDMS | 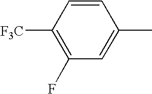 | 2 | 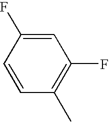 | H |
| Example 34 | —CH₃ | —TBDMS | 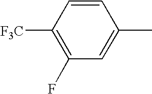 | 2 | 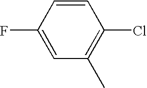 | H |

TABLE 2

| | ¹H NMR (300 MHz, CDCl₃) |
|---|---|
| Example 4 | 7.99 (d, 2H, J = 8.5 Hz), 7.67 (d, 2H, J = 8.2 Hz), 7.19 (m, 5H), |
| | 7.04 (d, 1H, J = 2.0 Hz), 6.96 (dd, 1H, J = 8.3, 2.4 Hz), 6.59 (d, 1H, J = 8.3 Hz), |
| | 4.19 (dd, 1H, J = 8.9, 6.0 Hz), 2.74 (m, 2H), 2.37 (m, 1H), |
| | 2.37 (m, 1H), 2.19 (m, 1H), 2.08 (s, 3H), 1.96 (s, 3H), 0.98 (s, 9H), |
| | 0.17 (s, 6H) |
| Example 5 | 7.97 (d, 2H, J = 8.5 Hz), 7.66 (d, 2H, J = 8.2 Hz), 7.19 (m, 5H), |
| | 7.05 (d, 1H, J = 2.0 Hz), 6.96 (dd, 1H, J = 8.3, 2.4 Hz), 6.60 (d, 1H, J = 8.3 Hz), |
| | 4.24 (dd, 1H, J = 8.6, 5.9 Hz), 2.64 (t, 2H, J = 7.7 Hz), |
| | 2.09 (m, 1H), 2.08 (s, 3H), 2.02 (s, 3H), 1.89 (m, 1H), 1.75 (m, 2H), |
| | 1.00 (s, 9H), 0.17 (s, 6H) |
| Example 6 | 7.98 (d, 2H, J = 8.0 Hz), 7.66 (d, 2H, J = 8.3 Hz), 7.19 (m, 5H), |
| | 7.07 (d, 1H, J = 1.8 Hz), 6.98 (dd, 1H, J = 8.2, 2.3 Hz), 6.61 (d, 1H, J = 8.3 Hz), |
| | 4.23 (dd, 1H, J = 9.1, 5.9 Hz), 2.58 (m, 2H), 2.09 (s, 3H), |
| | 2.06 (m, 1H), 2.04 (s, 3H), 1.89 (m, 1H), 1.62 (m, 2H), 1.46 (m, 2H), |
| | 0.98 (s, 9H), 0.17 (s, 6H) |
| Example 7 | 7.98 (d, 2H, J = 7.8 Hz), 7.66 (d, 2H, J = 8.2 Hz), 7.19 (m, 5H), |
| | 7.06 (d, 1H, J = 2.2 Hz), 6.97 (dd, 1H, J = 8.3, 2.3 Hz), 6.60 (d, 1H, J = 8.3 Hz), |
| | 4.22 (dd, 1H, J = 9.1, 5.9 Hz), 2.58 (t, 2H, J = 7.5 Hz), |
| | 2.09 (s, 3H), 2.08 (m, 1H), 2.02 (s, 3H), 1.85 (m, 1H), 1.60 (m, 2H), |
| | 1.38 (m, 4H), 0.98 (s, 9H), 0.17 (s, 6H) |
| Example 8 | 7.98 (d, 2H, J = 8.1 Hz), 7.66 (d, 2H, J = 8.2 Hz), 7.20 (m, 5H), |
| | 7.07 (d, 1H, J = 1.9 Hz), 6.98 (dd, 1H, J = 8.3, 2.3 Hz), 6.60 (d, 1H, J = 8.3 Hz), |
| | 4.23 (dd, 1H, J = 9.1, 5.9 Hz), 2.58 (t, 2H, J = 7.5 Hz), |
| | 2.09 (s, 3H), 2.08 (m, 1H), 2.04 (s, 3H), 1.84 (m, 1H), 1.60 (m, 2H), |
| | 1.35 (m, 8H), 0.98 (s, 9H), 0.17 (s, 6H) |
| Example 9 | 7.98 (d, 2H, J = 7.5 Hz), 7.66 (d, 2H, J = 8.4 Hz), 7.21 (m, 5H), |
| | 7.07 (d, 1H, J = 2.1 Hz), 6.98 (dd, 1H, J = 8.2, 2.2 Hz), 6.61 (d, 1H, J = 8.3 Hz), |
| | 4.24 (dd, 1H, J = 9.1, 7.5 Hz), 2.58 (t, 2H, J = 7.5 Hz), |
| | 2.09 (s, 3H), 2.08 (m, 1H), 2.04 (s, 3H), 1.83 (m, 1H), 1.59 (m, 4H), |
| | 1.25 (m, 12H), 0.98 (s, 9H), 0.17 (s, 6H) |
| Example10 | 7.98 (d, 2H, J = 8.1 Hz), 7.65 (d, 2H, J = 8.2 Hz), 7.11 (m, 4H), |
| | 6.90 (m, 1H), 6.63 (d, 1H, J = 8.3 Hz), 4.78 (dd, 1H, J = 8.8, 6.6 Hz), |
| | 3.39 (m, 2H), 2.01 (s, 3H), 1.91 (s, 3H), 0.98 (s, 9H), 0.17 (s, 6H) |
| Example11 | 7.98 (d, 2H, J = 8.1 Hz), 7.67 (d, 2H, J = 8.2 Hz), 7.11 (d, 2H, J = 1.7 Hz), |
| | 7.03 (dd, 1H, J = 8.3, 2.0 Hz), 6.72 (t, 2H, J = 8.0 Hz), |
| | 6.64 (d, 1H, J = 8.3 Hz), 4.45 (dd, 1H, J = 9.3, 5.8 Hz), 3.15 (m, 2H), |
| | 2.12 (s, 3H), 1.96 (s, 3H), 0.99 (s, 9H), 0.18 (s, 6H) |
| Example12 | 7.99 (d, 2H, J = 8.1 Hz), 7.67 (d, 2H, J = 8.2 Hz), 7.11 (s, 1H), |
| | 7.04 (dd, 1H, J = 8.3, 2.0 Hz), 6.64 (d, 1H, J = 8.3 Hz), 4.72 (t, 1H, J = 7.9 Hz), |
| | 3.37 (m, 2H), 2.11 (s, 6H), 0.98 (s, 9H), 0.18 (s, 6H) |
| Example13 | 7.98 (d, 2H, J = 8.1 Hz), 7.08 (m, 6H), 6.64 (d, 1H, J = 8.3 Hz), |
| | 4.48 (dd, 1H, J = 9.6, 5.4 Hz), 3.21 (m, 2H), 2.11 (s, 3H), 1.86 (s, 3H), |
| | 0.98 (s, 9H), 0.18 (s, 6H) |
| Example14 | 7.98 (d, 2H, J = 8.0 Hz), 7.66 (d, 2H, J = 8.2 Hz), 7.52 (d, 2H, J = 8.2 Hz), |
| | 7.19 (d, 2H, J = 8.2 Hz), 7.12 (s, 1H), 7.03 (dd, 1H, J = 8.2, |
| | 2.0 Hz), 6.64 (d, 1H, J = 8.3 Hz), 4.49 (dd, 1H, J = 9.5, 5.6 Hz), |
| | 3.28 (m, 2H), 2.11 (s, 3H), 1.88 (s, 3H), 0.98 (s, 9H), 0.18 (s, 6H) |
| Example15 | 7.97 (d, 2H, J = 8.1 Hz), 7.69 (m, 5H), 7.55 (s, 1H), 7.41 (m, 2H), |
| | 7.20 (d, 2H, J = 8.2 Hz), 7.13 (s, 1H), 7.07 (d, 1H, J = 8.2, Hz), |
| | 6.63 (d, 1H, J = 8.3 Hz), 4.64 (dd, 1H, J = 9.6, 5.5 Hz), 3.28 (m, 2H), |
| | 2.10 (s, 3H), 1.84 (s, 3H), 0.98 (s, 9H), 0.18 (s, 6H) |
| Example16 | 7.98 (d, 2H, J = 8.0 Hz), 7.67 (d, 2H, J = 8.2 Hz), 7.48 (d, 2H, J = 5.0 Hz), |
| | 7.20 (d, 2H, J = 8.0 Hz), 7.12 (s, 1H), 7.05 (d, 1H, J = 8.3 Hz), |
| | 6.64 (d, 1H, J = 8.3 Hz), 4.51 (dd, 1H, J = 9.6, 5.6 Hz), 3.28 (m, |
| | 2H), 2.11 (s, 3H), 1.87 (s, 3H), 0.98 (s, 9H), 0.18 (s, 6H) |
| Example17 | 7.98 (d, 2H, J = 8.1 Hz), 7.66 (d, 2H, J = 8.2 Hz), 7.13 (d, 1H, J = 1.8 Hz), |
| | 7.10 (s, 1H), 7.05 (dd, 1H, J = 8.3, 2.3 Hz), 6.63 (d, 1H, J = 8.3 Hz), |
| | 4.53 (dd, 1H, J = 9.7, 5.4 Hz), 3.69 (s, 6H), 3.17 (m, 2H), |
| | 2.11 (s, 3H), 1.92 (s, 3H), 0.98 (s, 9H), 0.18 (s, 6H) |
| Example 18 | 7.98 (d, 2H, J = 8.1 Hz), 7.66 (d, 2H, J = 8.2 Hz), 7.11 (d, 1H, J = 1.9 Hz), |
| | 7.04 (dd, 1H, J = 8.3, 2.4 Hz), 6.64 (d, 1H, J = 8.3 Hz), |
| | 4.66 (dd, 1H, J = 8.0, 8.0 Hz), 3.31 (m, 2H), 2.11 (s, 3H), 2.08 (s, 3H), |
| | 0.98 (s, 9H), 0.18 (s, 6H) |
| Example19 | 7.98 (d, 2H, J = 8.0 Hz), 7.65 (d, 2H, J = 8.4 Hz), 7.33 (d, 2H, J = 8.3 Hz), |
| | 7.11 (d, 1H, J = 2.1 Hz), 7.04 (dd, 1H, J = 8.3, 2.3 Hz), |
| | 6.95 (d, 2H, J = 8.3 Hz), 6.63 (d, 1H, J = 8.3 Hz), 4.47 (dd, 1H, J = 9.7, |
| | 5.4 Hz), 3.16 (m, 2H), 2.11 (s, 3H), 1.87 (s, 3H), 0.98 (s, 9H), 0.18 (s, |
| | 6H) |
| Example20 | 8.00 (d, 2H, J = 8.1 Hz), 7.66 (d, 2H, J = 8.3 Hz), 7.46 (d, 1H, J = 7.8 Hz), |
| | 7.32 (q, 1H, J = 4.7 Hz), 7.18 (d, 1H, J = 7.4 Hz), 7.12 (d, |
| | 1H, J = 1.7 Hz), 7.04 (dd, 1H, J = 8.2, 2.2 Hz), 6.62 (d, 1H, J = 8.3 Hz), |
| | 4.70 (dd, 1H, J = 8.4, 7.1 Hz), 3.43 (m, 2H), 2.10 (s, 3H), 1.84 (s, |
| | 3H), 0.98 (s, 9H), 0.17 (s, 6H) |
| Example21 | 7.98 (d, 2H, J = 8.1 Hz), 7.65 (d, 2H, J = 8.3 Hz), 7.14 (m, 2H), |
| | 7.05 (dd, 1H, J = 8.3, 2.4 Hz), 6.80 (t, 1H, J = 7.8), 6.63 (d, 1H, J = 8.3), |
| | 4.71 (dd, 1H, J = 9.1, 6.6 Hz), 3.29 (m, 2H), 2.11 (s, 3H), 1.97 (s, |
| | 3H), 0.98 (s, 9H), 0.17 (s, 6H) |
| Example 22 | 8.00 (d, 2H, J = 8.3 Hz), 7.66 (d, 2H, J = 8.4 Hz), 7.25 (d, 2H, J = 7.5 Hz), |
| | 7.17 (d, 1H, J = 2.1 Hz), 7.09 (m, 2H), 6.63 (d, 1H, J = 8.3 Hz), |
| | 4.84 (dd, 1H, J = 8.7, 6.5 Hz), 3.53 (m, 2H), 2.11 (s, 3H), 1.84 (s, |
| | 3H), 0.98 (s, 9H), 0.17 (s, 6H) |
| Example 23 | 7.98 (d, 2H, J = 8.1 Hz), 7.66 (d, 2H, J = 8.3 Hz), 7.13 (d, 1H, J = 2.1 Hz), |
| | 7.04 (m, 2H), 6.73 (m, 2H), 6.63 (d, 1H, J = 8.3 Hz), |
| | 4.59 (dd, 1H, J = 9.4, 6.0 Hz), 3.22 (m, 2H), 2.11 (s, 3H), 1.94 (s, 3H), |
| | 0.98 (s, 9H), 0.18 (s, 6H) |
| Example 24 | 7.99 (d, 2H, J = 8.1 Hz), 7.66 (d, 2H, J = 8.3 Hz), 7.27 (m, 1H), |
| | 7.16 (d, 1H, J = 2.1 Hz), 7.09 (dd, 1H, J = 8.3, 2.4 Hz), 6.81 (m, 2H), |
| | 6.64 (d, 1H, J = 8.3 Hz), 4.72 (dd, 1H, J = 9.3, 5.9 Hz), 3.29 (m, 2H), |
| | 2.12 (s, 3H), 1.93 (s, 3H), 0.98 (s, 9H), 0.18 (s, 6H) |
| Example 25 | 8.11 (d, 1H, J = 3.8 Hz), 8.00 (d, 2H, J = 8.0 Hz), 7.64 (d, 2H, J = 8.2 Hz), |
| | 7.42 (m, 1H), 7.21 (s, 1H), 7.14 (dd, 1H, J = 8.2, 2.1 Hz), |
| | 7.03 (d, 2H, J = 8.6 Hz), 6.73 (d, 2H, J = 8.6 Hz), 6.68 (d, 1H, J = 8.3 Hz), |
| | 6.53 (m, 1H), 6.47 (d, 1H, J = 8.7 Hz), 5.04 (d, 1H, J = 3.1 Hz), |
| | 4.57 (d, 1H, J = 3.4 Hz), 4.09 (t, 2H, J = 5.6 Hz), 3.92 (t, 2H, J = 5.4 Hz), |
| | 3.14 (br s, 1H), 3.09 (s, 3H), 2.14 (s, 3H), 1.88 (s, 3H), |
| | 0.99 (s, 9H), 0.19 (s, 6H) |
| Example 26 | 8.06 (d, 2H, J = 8.1 Hz), 7.68 (d, 2H, J = 8.0 Hz), 7.15 (m, 3H), |
| | 7.12 (m, 1H), 6.96 (m, 1H), 6.66 (d, 1H, J = 8.3 Hz), 4.44 (d, 1H, J = 6.8 Hz), |
| | 4.32 (m, 1H), 3.01 (m, 2H), 2.61 (d, 1H, J = 3.7 Hz), 2.25 (s, 3H), |
| | 2.09 (s, 3H), 1.00 (s, 9H), 0.19 (s, 6H) |
| Example 27 | 8.01 (d, 2H, J = 8.1 Hz), 7.66 (d, 2H, J = 8.3 Hz), 7.20 (m, 6H), |
| | 6.71 (d, 1H, J = 8.2 Hz), 5.09 (m, 1H), 4.63 (d, 1H, J = 3.2 Hz), 2.91 (d, |
| | 1H, J = 2.5 Hz), 2.16 (s, 3H), 1.86 (s, 3H), 1.00 (s, 9H), 0.21 (s, 6H) |
| Example 28 | 7.59-7.74 (m, 3H), 7.02-7.24 (m, 7H), 6.63 (d, 1H, J = 8.3 Hz), |
| | 4.52 (dd, 1H, J = 9.8, 5.3 Hz), 3.23 (m, 2H), 2.11 (s, 3H), 1.84 (s, 3H), |
| | 0.99 (s, 9H), 0.18 (s, 6H) |
| Example 29 | 7.61-7.75 (m, 3H), 7.11 (d, 1H, J = 1.8 Hz), 7.03 (dd, 1H, J = 8.2, 2.3 Hz), |
| | 6.72 (m, 2H), 6.64 (d, 1H, J = 8.3 Hz), 4.45 (dd, 1H, J = 9.3, 5.8 Hz), |
| | 3.15 (m, 2H), 2.12 (s, 3H), 1.96 (s, 3H), 0.99 (s, 9H), 0.18 (s, 6H) |
| Example 30 | 7.60-7.77 (m, 3H), 7.46 (d, 1H, J = 7.8 Hz), 7.33 (m, 1H), 7.17 (t, 1H, |
| | J = 9.1 Hz), 7.12 (d, 1H, J = 1.8 Hz), 7.04 (dd, 1H, J = 8.2, 2.3 Hz), |
| | 6.63 (m, 2H), 6.63 (d, 1H, J = 8.3 Hz), 4.69 (dd, 1H, J = 8.5, 6.9 Hz), |
| | 3.43 (m, 2H), 2.11 (s, 3H), 1.84 (s, 3H), 0.99 (s, 9H), 0.18 (s, 6H) |
| Example 31 | 7.59-7.74 (m, 3H), 7.10-7.17 (m, 2H), 7.05 (dd, 1H, J = 8.2, 2.3 Hz), |
| | 6.80 (m, 1H), 6.63 (d, 1H, J = 8.3 Hz), 4.70 (dd, 1H, J = 9.2, 6.6 Hz), |
| | 3.29 (m, 2H), 2.11 (s, 3H), 1.97 (s, 3H), 0.99 (s, 9H), 0.18 (s, 6H) |
| Example 32 | 7.60-7.77 (m, 3H), 7.23-7.26 (m, 2H), 7.17 (d, 1H, J = 1.8 Hz), |
| | 7.06-7.12 (m, 2H), 6.63 (d, 1H, J = 8.3 Hz), 4.84 (dd, 1H, J = 8.8, 6.4 Hz), |
| | 3.52 (m, 2H), 2.12 (s, 3H), 1.83 (s, 3H), 0.99 (s, 9H), 0.18 (s, 6H) |
| Example 33 | 7.60-7.75 (m, 3H), 7.12 (d, 1H, J = 1.8 Hz), 6.97-7.06 (m, 2H), |
| | 6.69-6.78 (m, 2H), 6.63 (d, 1H, J = 8.3 Hz), 4.59 (dd, 1H, J = 9.4, 6.0 Hz), |
| | 3.21 (m, 2H), 2.11 (s, 3H), 1.94 (s, 3H), 0.99 (s, 9H), 0.18 (s, 6H) |
| Example 34 | 7.61-7.76 (m, 3H), 7.27 (m, 1H), 7.15 (d, 1H, J = 1.8 Hz), 7.07 (dd, |
| | 1H, J = 8.2, 2.3 Hz), 6.86 (m, 1H), 6.77 (dd, 1H, J = 8.9, 3.0 Hz), |
| | 6.65 (d, 1H, J = 8.3 Hz), 4.72 (dd, 1H, J = 9.2, 5.9 Hz), 3.29 (m, 2H), |
| | 2.12 (s, 3H), 1.93 (s, 3H), 0.99 (s, 9H), 0.18 (s, 6H) |

### Example 35

### Preparation of 4-[1-[2-[4-(trifluoromethyl)phenyl]-4-methylthiazol-5-yl]-6-phenylhexylthio]-2-methylphenol (VII) [Step D]

394 mg (0.6 mmol) of 5-[1-[3-methyl-4-(tert-butyldimethylsilyloxy)phenylthio]-6-phenylhexyl]-2-[4-(trifluoromethyl)phenyl]-4-methylthiazol was completely dissolved in 10 ml of tetrahydrofuran. To the solution, 1.5 ml (2.5 equivalents) of tetrabutylammonium fluoride (TBAF)(1 M-tetrahydrofuran solution) was slowly added at room temperature. After reaction for 30 minutes, the reaction product was extracted with aqueous ammonium chloride solution and ethyl acetate, and the organic layer was dried over magnesium sulfate. After filtration, the solvent was removed by distillation under reduced pressure, and the residue was purified by silica gel column chromatography, thus obtaining 306 mg (94% yield) of the title compound.

¹H NMR (300 MHz, CDCl₃) δ7.98 (d, 2H, J=8.0 Hz), 7.66 (d, 2H, J=8.2 Hz), 7.19 (m, 5H), 7.09 (d, 1H, J=1.5 Hz), 6.93 (dd, 1H, J=7.8, 1.9 Hz), 6.57 (d, 1H, J=8.2 Hz), 4.20 (dd, 1H, J=9.1, 5.9 Hz), 2.58 (t, 2H, J=7.5 Hz), 2.18 (s, 3H), 2.04 (m, 1H), 1.97 (s, 3H), 1.85 (m, 1H), 1.60 (m, 2H), 1.39 (m, 4H).

### Examples 36 to 38

Compounds shown in Table 3 below were prepared according to the method of Example 35, and the NMR data of the prepared compounds are shown in Table 4 below.

TABLE 3

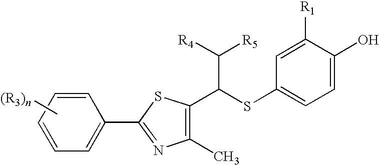

| | R₁ | 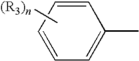 | n | R₄ | R₅ |
|---|---|---|---|---|---|
| Ex-am-ple35 | —CH₃ | 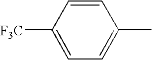 | 1 | 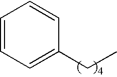 | H |
| Ex-am-ple36 | —CH₃ | 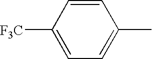 | 1 | 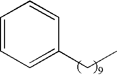 | H |
| Ex-am-ple37 | —CH₃ | 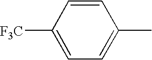 | 1 | 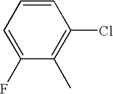 | H |
| Ex-am-ple38 | —CH₃ | 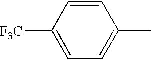 | 1 | 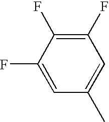 | H |

TABLE 4

| | ¹H NMR (300 MHz, CDCl₃) |
|---|---|
| Example 35 | 7.98 (d, 2H, J = 8.0 Hz), 7.66 (d, 2H, J = 8.2 Hz), 7.19 (m, 5H), |
| | 7.09 (d, 1H, J = 1.5 Hz), 6.93 (dd, 1H, J = 7.8, 1.9 Hz), 6.57 (d, 1H, J = 8.2 Hz), |
| | 4.20 (dd, 1H, J = 9.1, 5.9 Hz), 2.58 (t, 2H, J = 7.5 Hz), |
| | 2.18 (s, 3H), 2.04 (m, 1H), 1.97 (s, 3H), 1.85 (m, 1H), 1.60 (m, 2H), |
| | 1.39 (m, 4H) |
| Example 36 | 7.98 (d, 2H, J = 8.0 Hz), 7.66 (d, 2H, J = 8.3 Hz), 7.21 (m, 5H), |
| | 7.09 (d, 1H, J = 1.5 Hz), 6.96 (dd, 1H, J = 8.0, 1.9 Hz), 6.59 (d, 1H, J = 8.2 Hz), |
| | 5.21 (s, 1H), 4.22 (dd, 1H, J = 9.1, 5.9 Hz), 2.58 (t, 2H, J = 7.5 Hz), |
| | 2.14 (s, 3H), 2.02 (s, 3H), 2.00 (m, 1H), 1.83 (m, 1H), |
| | 1.58 (m, 4H), 1.28 (m, 12H) |
| Example 37 | 7.97 (d, 2H, J = 8.1 Hz), 7.64 (d, 2H, J = 8.2 Hz), 7.14 (m, 3H), |
| | 6.98 (dd, 1H, J = 8.2, 2.1 Hz), 6.90 (m, 1H), 6.55 (d, 1H, J = 8.3 Hz), |
| | 4.74 (dd, 1H, J = 8.7, 6.8 Hz), 3.40 (m, 2H), 2.18 (s, 3H), 1.85 (s, 3H) |
| Example 38 | 7.97 (d, 2H, J = 8.2 Hz), 7.67 (d, 2H, J = 8.2 Hz), 7.13 (d, 2H, J = 1.4 Hz), |
| | 6.91 (dd, 1H, J = 8.2, 1.9 Hz), 6.74 (t, 2H, J = 8.1 Hz), |
| | 6.54 (d, 1H, J = 8.2 Hz), 4.41 (dd, 1H, J = 9.3, 5.9 Hz), 3.16 (m, 2H), |
| | 2.16 (s, 3H), 1.87 (s, 3H) |

### Example 39

### Preparation of 4-[2-(2-chloro-6-fluorophenyl)-1-[2-[4-(trifluoromethyl)phenyl]-4-methylthiazol-5-yl]ethylthio]-2-methylphenol (VII) from Compound of Formula II [Step E]

In a nitrogen atmosphere, 585 mg (2.5 mmol) of 4-iodo-2-methylphenol was dissolved in 35 ml of anhydrous tetrahydrofuran and maintained at a temperature of 0° C. To the solution, 1.3 ml (1.0 equivalent) of isopropyl magnesium chloride (2 M-ether solution) was slowly added and the mixture was allowed to react for 10 minutes. After the reaction solution was sufficiently cooled to −78° C., 3.0 ml (2.0 equivalents) of tert-butyllithium (1.7 M-heptane solution) was added dropwise thereto, and the mixture was allowed to react for 20 minutes. To the reaction product, 80 mg (2.5 mmol, 1.0 equivalent) of solid phase sulfur was added at a time, and the reaction mixture was allowed to react until it reached a temperature of 15° C. After 40 minutes, 730 mg (2.5 mmol, 1.0 equivalent) of 5-chloromethyl-4-methyl-2-[(4-trifluoromethyl)phenyl]-thiazole of Formula IV dissolved in 3 ml of anhydrous THF was added to the reaction product at the same temperature. After reaction for an additional time of about 20 minutes, the reaction material was sufficiently cooled to −78° C. Then, 3.0 ml (2.0 equivalents) of tert-butyllithium (1.7 M-heptane solution) was added dropwise to the reaction solution, and when the reaction solution turned a blue color, 345 μl (2.5 mmol) of 2-chloro-6-fluorobenzyl bromide was added thereto at the same temperature. The mixture was allowed to react while elevating the temperature slowly to room temperature. After 20 minutes, 30 ml of aqueous ammonium chloride solution was added to terminate the reaction. The organic layer was separated and dried over magnesium sulfate. After filtration, the solvent was removed by distillation under reduced pressure. The residue was purified by silica gel column chromatography using hexane/ethyl acetate (v/v=3/1), thus obtaining 1.12 g (83% yield) of the title compound.

¹H NMR (300 MHz, CDCl₃) δ7.97 (d, 2H, J=8.1 Hz), 7.64 (d, 2H, J=8.2 Hz), 7.14 (m, 3H), 6.98 (dd, 1H, J=8.2, 2.1 Hz), 6.90 (m, 1H), 6.55 (d, 1H, J=8.3 Hz), 4.74 (dd, 1H, J=8.7, 6.8 Hz), 3.40 (m, 2H), 2.18 (s, 3H), 1.85 (s, 3H).

### Examples 40 to 41

Compounds shown in Table 5 below were prepared according to the method of Example 39, and the NMR data of the prepared compounds are shown in Table 6 below.

TABLE 5

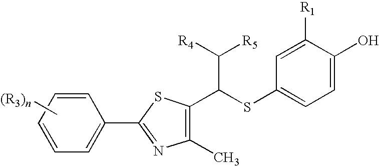

| | R₁ | 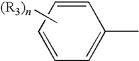 | n | R₄ | R₅ |
|---|---|---|---|---|---|
| Ex-am-ple39 | —CH₃ | 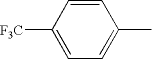 | 1 | 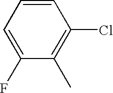 | H |
| Ex-am-ple40 | CH₃ | 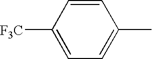 | 1 | 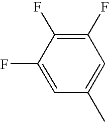 | H |
| Ex-am-ple41 | —CH₃ | 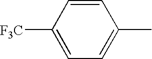 | 1 | 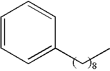 | OH |

TABLE 6

| | ¹H NMR (300 MHz, CDCl₃) |
|---|---|
| Example 39 | 7.97 (d, 2H, J = 8.1 Hz), 7.64 (d, 2H, J = 8.2 Hz), 7.14 (m, 3H), |
| | 6.98 (dd, 1H, J = 8.2, 2.1 Hz), 6.90 (m, 1H), 6.55 (d, 1H, J = 8.3 Hz), |
| | 4.74 (dd, 1H, J = 8.7, 6.8 Hz), 3.40 (m, 2H), 2.18 (s, 3H), 1.85 (s, 3H) |
| Example40 | 7.97 (d, 2H, J = 8.2 Hz), 7.67 (d, 2H, J = 8.2 Hz), 7.13 (d, 2H, J = 1.4 Hz), |
| | 6.91 (dd, 1H, J = 8.2, 1.9 Hz), 6.74 (t, 2H, J = 8.1 Hz), |
| | 6.54 (d, 1H, J = 8.2 Hz), 4.41 (dd, 1H, J = 9.3, 5.9 Hz), 3.16 (m, 2H), |
| | 2.16 (s, 3H), 1.87 (s, 3H) |
| Example41 | 8.02 (d, 2H, J = 8.2 Hz), 7.65 (d, 2H, J = 8.2 Hz), 7.22 (m, 7H), |
| | 6.64 (d, 1H, J = 8.3 Hz), 5.68 (br s, 1H), 4.40 (d, 1H, J = 2.9 Hz), |
| | 3.96 (m, 1H), 2.85 (d, 1H, J = 3.8 Hz), 2.57 (t, 2H, J = 7.4 Hz), 2.21 (s, |
| | 3H), 2.10 (s, 3H), 1.59-1.21 (m, 16H) |

### Example 42

### Preparation of 2-[4-[1-[2-[4-(trifluoromethyl)phenyl]-4-methylthiazol-5-yl]-4-phenylbutylthio]-2-methylphenoxy]acetic acid ethyl ester of Formula IX [Step F]

At room temperature, 205 mg (0.4 mmol) of 2-methyl-4-[1-[4-methyl-2-(4-trifluoromethyl-phenyl)thiazol-5-yl]-4-phenyl-butylsulfanyl]phenol was well mixed with 10 ml of acetone containing 5% water and 127 mg (0.92 mmol, 2.3 equivalents) of potassium carbonate. To the solution, 67 μl (0.6 mmol, 1.5 equivalents) of bromoacetic acid ethyl ester was added and the mixture was strongly stirred for 4 hours. After completion of the reaction, the reaction product was extracted with aqueous salt solution and ethyl acetate and the organic layer was dried over magnesium sulfate. After filtration, the solvent was removed by distillation under reduced pressure, and the residue was purified by silica gel column chromatography using hexane/ethyl acetate (v/v=5:1), thus obtaining 230 mg (96% yield) of the title compound.

¹H NMR (300 MHz, CDCl₃) δ7.98 (d, 2H, J=8.2 Hz), 7.66 (d, 2H, J=8.5 Hz), 7.19 (m, 6H), 7.01 (dd, 1H, J=8.4, 2.2 Hz), 6.52 (d, 1H, J=8.4), 4.59 (s, 2H), 4.23 (m, 3H), 2.63 (t, 2H, J=8.4 Hz), 2.19 (s, 3H), 2.08 (m, 1H), 2.02 (s, 3H), 1.90 (m, 1H), 1.73 (m, 2H), 1.27 (t, 3H, J=7.2 Hz).

¹³C NMR (75.5 MHz, CDCl₃) δ168.9, 163.3, 156.9, 151.2, 141.8, 137.8, 137.1, 137.0, 133.8, 131.6 (q, J=33 Hz), 128.6, 128.6, 128.3, 126.5, 126.2, 126.0 (q, J=4 Hz), 124.6, 111.5, 65.7, 61.6, 47.5, 37.3, 35.6, 29.6, 16.3, 15.2, 14.3.

### Example 43

### Preparation of 2-[4-[2-(2-chloro-6-fluorophenyl)-1-[2-[4-(trifluoromethyl)phenyl]-4-methylthiazol-5-yl]ethylthio]-2-methylphenoxy]acetic acid allyl ester of Formula Ix [Step F]

At room temperature, 200 mg (0.37 mmol) of 4-(2-(2-chloro-6-fluorophenyl)-1-(2-(4-(trifluoromethyl)phenyl)-4-methylthiazol-5-yl)ethylthio)-2-methylphenol prepared in Example 37 was well mixed with 10 ml of acetone containing 5% water and 102 mg (0.74 mmol, 2 equivalents) of potassium carbonate. To the solution, 73 mg (0.40 mmol, 1.1 equivalents) of bromoacetic acid allyl ester was added and the mixture was strongly stirred for 4 hours. After completion of the reaction, the reaction product was extracted with aqueous salt solution and ethyl acetate, and the organic layer was dried over magnesium sulfate. After filtration, the solvent was removed by distillation under reduced pressure, and the residue was purified by silica gel column chromatography using a mixed solvent of hexane/ethyl acetate (v/v=5:1), thus obtaining 221 mg (94% yield) of the title compound.

¹H NMR (300 MHz, CDCl₃) δ 7.60-7.76 (m, 3H), 7.11-7.17 (m, 4H), 6.90 (m, 1H), 6.55 (d, 1H, J=8.4 Hz), 5.89 (m, 1H), 5.34 (m, 1H), 5.24 (m, 1H), 4.79 (dd, 1H, J=8.8, 6.6 Hz), 4.68 (m, 2H), 4.59 (s, 2H), 3.38 (m, 2H), 2.20 (s, 3H), 1.90 (s, 3H).

### Examples 44 to 67

Compounds shown in Table 7 below were prepared according to the method of Example 42, and the NMR data of the prepared compounds are shown in Table 8 below.

TABLE 7

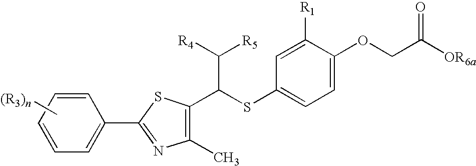

| | R₁ | 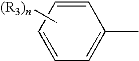 | n | R₄ | R₅ | R₆ₐ |
|---|---|---|---|---|---|---|
| Example 42 | —CH₃ | 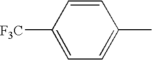 | 1 | 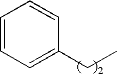 | H | —CH₂CH₃ |
| Example 43 | —CH₃ | 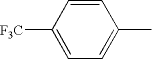 | 1 | 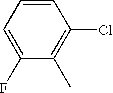 | H | —CH₂CH═CH₂ |
| Example 44 | —CH₃ | 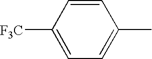 | 1 | 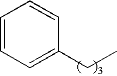 | H | —CH₂CH₃ |
| Example 45 | —CH₃ | 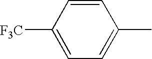 | 1 | 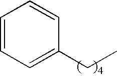 | H | —CH₂CH₃ |
| Example 46 | —CH₃ | 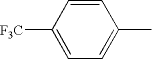 | 1 | 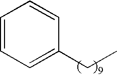 | H | —CH₂CH₃ |
| Example 47 | —CH₃ | 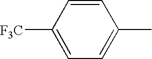 | 1 | 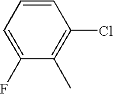 | H | —CH₂CH₃ |
| Example 48 | —CH₃ | 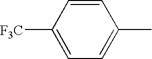 | 1 | 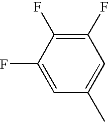 | H | —CH₂CH₃ |
| Example 49 | —CH₃ | 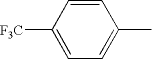 | 1 | 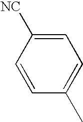 | H | —CH₂CH₃ |
| Example 50 | —CH₃ | 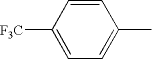 | 1 | 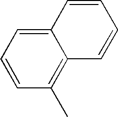 | H | —CH₂CH₃ |
| Example 51 | —CH₃ | 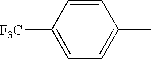 | 1 | 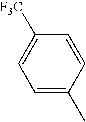 | H | —CH₂CH₃ |
| Example 52 | —CH₃ | 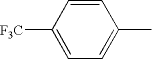 | 1 | 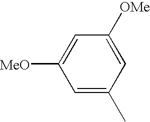 | H | —CH₂CH₃ |
| Example 53 | —CH₃ | 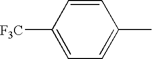 | 1 | 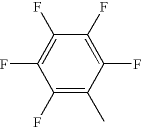 | H | —CH₂CH₃ |
| Example 54 | —CH₃ | 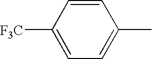 | 1 | 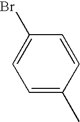 | H | —CH₂CH₃ |
| Example 55 | —CH₃ | 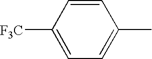 | 1 | 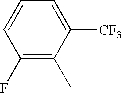 | H | —CH₂CH₃ |
| Example 56 | —CH₃ | 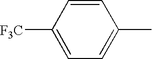 | 1 | 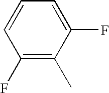 | H | —CH₂CH₃ |
| Example 57 | —CH₃ | 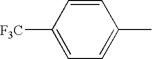 | 1 | 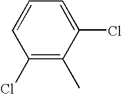 | H | —CH₂CH₃ |
| Example 58 | —CH₃ | 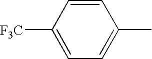 | 1 | 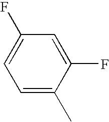 | H | —CH₂CH₃ |
| Example 59 | —CH₃ | 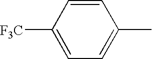 | 1 | 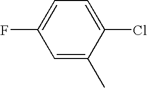 | H | —CH₂CH₃ |
| Example 60 | —CH₃ | 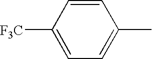 | 1 | 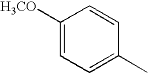 | OH | —CH₂CH₃ |
| Example 61 | —CH₃ | 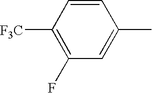 | 2 | 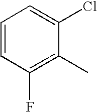 | H | —CH₂CH₃ |
| Example 62 | —CH₃ | 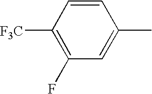 | 2 | 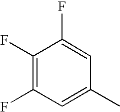 | H | —CH₂CH₃ |
| Example 63 | —CH₃ | 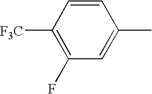 | 2 | 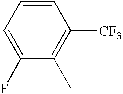 | H | —CH₂CH₃ |
| Example 64 | —CH₃ | 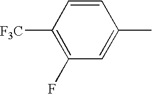 | 2 | 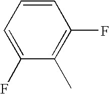 | H | —CH₂CH₃ |
| Example 65 | —CH₃ | 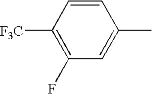 | 2 | 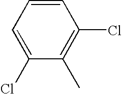 | H | —CH₂CH₃ |
| Example 66 | —CH₃ | 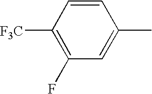 | 2 | 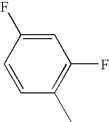 | H | —CH₂CH₃ |
| Example 67 | —CH₃ | 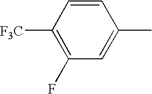 | 2 | 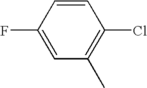 | H | —CH₂CH₃ |

TABLE 8

| | ¹H NMR (300 MHz, CDCl₃) |
|---|---|
| Example 42 | 7.98 (d, 2H, J = 8.2 Hz), 7.66 (d, 2H, J = 8.5 Hz), 7.19 (m, 6H), |
| | 7.01 (dd, 1H, J = 8.4, 2.2 Hz), 6.52 (d, 1H, J = 8.4), 4.59 (s, 2H), |
| | 4.23 (m, 3H), 2.63 (t, 2H, J = 8.4 Hz), 2.19 (s, 3H), 2.08 (m, 1H), |
| | 2.02 (s, 3H), 1.90 (m, 1H), 1.73 (m, 2H), 1.27 (t, 3H, J = 7.2 Hz) |
| Example 43 | 7.60-7.76 (m, 3H), 7.11-7.17 (m, 4H), 6.90 (m, 1H), 6.55 (d, 1H, J = 8.4 Hz), |
| | 5.89 (m, 1H), 5.34 (m, 1H), 5.24 (m, 1H), 4.79 (dd, 1H, J = 8.8, |
| | 6.6 Hz), 4.68 (m, 2H), 4.59 (s, 2H), 3.38 (m, 2H), 2.20 (s, 3H), |
| | 1.90 (s, 3H) |
| Example 44 | 7.99 (d, 2H, J = 8.2 Hz), 7.66 (d, 2H, J = 8.5 Hz), 7.15 (m, 6H), |
| | 7.03 (dd, 1H, J = 8.4, 2.2 Hz), 6.52 (d, 1H, J = 8.4 Hz), 4.59 (s, |
| | 2H), 4.24 (m, 3H), 2.58 (m, 2H), 2.19 (s, 3H), 2.05 (m, 1H), 2.03 (s, |
| | 3H), 1.90 (m, 1H), 1.61 (m, 2H), 1.46 (m, 2H), 1.27 (t, 3H, J = 7.2 Hz) |
| Example 45 | 7.98 (d, 2H, J = 8.1 Hz), 7.66 (d, 2H, J = 8.5 Hz), 7.19 (m, 6H), |
| | 7.03 (dd, 1H, J = 8.4, 2.2 Hz), 6.52 (d, 1H, J = 8.4 Hz), 4.59 (s, |
| | 2H), 4.23 (m, 3H), 2.58 (t, 2H, J = 7.6 Hz), 2.19 (s, 3H), 2.03 (m, |
| | 1H), 2.02 (s, 3H), 1.85 (m, 1H), 1.45 (m, 6H), 1.27 (t, 3H, J = 7.1 Hz) |
| Example 46 | 7.99 (d, 2H, J = 8.4 Hz), 7.66 (d, 2H, J = 8.5 Hz), 7.19 (m, 6H), |
| | 7.03 (dd, 1H, J = 8.4, 2.2 Hz), 6.52 (d, 1H, J = 8.4 Hz), 4.59 (s, |
| | 2H), 4.23 (m, 3H), 2.58 (t, 2H, J = 7.5 Hz), 2.19 (s, 3H), 2.04 (s, |
| | 3H), 2.02 (m, 1H), 1.85 (m, 1H), 1.58 (m, 4H), 1.29 (m, 15H) |
| Example 47 | 7.99 (d, 2H, J = 8.1 Hz), 7.66 (d, 2H, J = 8.2 Hz), 7.14 (m, 4H), |
| | 6.90 (m, 1H), 6.54 (d, 1H, J = 8.4 Hz), 4.79 (dd, 1H, J = 8.7, 6.7 Hz), |
| | 4.59 (s, 2H), 4.23 (q, 2H, J = 7.1 Hz), 3.38 (m, 2H), 2.20 (s, |
| | 3H), 1.91 (s, 3H), 1.26 (t, 3H, J = 7.2 Hz) |
| Example 48 | 7.98 (d, 2H, J = 8.1 Hz), 7.67 (d, 2H, J = 8.2 Hz), 7.15 (s, 1H), |
| | 7.09 (dd, 1H, J = 8.4, 2.2 Hz), 6.73 (t, 1H, J = 7.6 Hz) 6.56 (d, 1H, J = 8.4 Hz), |
| | 4.60 (s, 2H), 4.46 (dd, 1H, J = 9.3, 5.8 Hz), 4.24 (q, 2H, J = 7.1 Hz), |
| | 3.69 (s, 6H), 3.15 (m, 2H), 2.21 (s, 3H), 1.95 (s, 3H), |
| | 1.26 (t, 3H, J = 7.2 Hz) |
| Example 49 | 7.98 (d, 2H, J = 8.1 Hz), 7.67 (d, 2H, J = 8.2 Hz), 7.52 (d, 2H, J = 8.2 Hz), |
| | 7.19 (d, 2H, J = 8.2 Hz), 7.15 (d, 2H, J = 1.5 Hz), 7.09 (dd, |
| | 1H, J = 8.4, 1.7 Hz), 6.56 (d, 1H, J = 8.5 Hz), 4.60 (s, 2H), |
| | 4.49 (dd, 1H, J = 9.5, 5.6 Hz), 4.23 (q, 2H, J = 7.1 Hz), 3.27 (m, 2H), |
| | 2.20 (s, 3H), 1.87 (s, 3H), 1.26 (t, 3H, J = 7.2 Hz) |
| Example 50 | 7.97 (d, 2H, J = 8.1 Hz), 7.74 (m, 1H), 7.67 (d, 2H, J = 8.2 Hz), |
| | 7.72 (d, 2H, J = 8.5 Hz), 7.66 (d, 2H, J = 8.3 Hz), 7.55 (s, 1H), |
| | 7.42 (m, 2H), 7.17 (m, 2H), 7.07 (dd, 1H, J = 8.4, 1.7 Hz), 6.54 (d, 1H, J = 8.5 Hz), |
| | 4.60 (s, 2H), 4.48 (dd, 1H, J = 9.5, 5.6 Hz), 4.22 (q, 2H, |
| | J = 7.1 Hz), 3.27 (m, 2H), 2.21 (s, 3H), 1.87 (s, 3H), 1.26 (t, 3H, J = 7.2 Hz) |
| Example 51 | 7.98 (d, 2H, J = 8.1 Hz), 7.67 (d, 2H, J = 8.2 Hz), 7.48 (d, 2H, J = 8.2 Hz), |
| | 7.20 (d, 2H, J = 8.2 Hz), 7.16 (d, 2H, J = 1.5 Hz), 7.09 (dd, |
| | 1H, J = 8.4, 1.7 Hz), 6.57 (d, 1H, J = 8.5 Hz), 4.59 (s, 2H), |
| | 4.52 (dd, 1H, J = 9.6, 5.5 Hz), 4.23 (q, 2H, J = 7.1 Hz), 3.19 (m, 2H), |
| | 2.20 (s, 3H), 1.86 (s, 3H), 1.26 (t, 3H, J = 7.2 Hz) |
| Example 52 | 7.97 (d, 2H, J = 8.1 Hz), 7.66 (d, 2H, J = 8.3 Hz), 7.16 (d, 1H, J = 1.6 Hz), |
| | 7.09 (dd, 1H, J = 8.4, 2.2 Hz), 6.54 (d, 1H, J = 8.4 Hz), |
| | 6.29 (t, 1H, J = 2.2 Hz) 6.23, (d, 2H, J = 2.2 Hz), 4.59 (s, 2H), |
| | 4.54 (dd, 1H, J = 9.7, 5.4 Hz), 4.23 (q, 2H, J = 7.1 Hz), 3.69 (s, 6H), |
| | 3.16 (m, 2H), 2.20 (s, 3H), 1.90 (s, 3H), 1.26 (t, 3H, J = 7.2 Hz) |
| Example53 | 7.99 (d, 2H, J = 8.3 Hz), 7.67 (d, 2H, J = 8.4 Hz), 7.14 (d, 1H, J = 2.0 Hz), |
| | 7.10 (dd, 1H, J = 8.4, 2.2 Hz), 6.56 (d, 1H, J = 8.4 Hz), |
| | 4.67 (dd, 1H, J = 8.0, 8.0 Hz), 4.60 (s, 2H), 4.23 (q, 2H, J = 7.1 Hz), |
| | 3.30 (m, 2H), 2.07 (s, 3H), 1.90 (s, 3H), 1.27 (t, 3H, J = 7.2 Hz) |
| Example54 | 7.98 (d, 2H, J = 8.3 Hz), 7.66 (d, 2H, J = 8.3 Hz), 7.34 (d, 2H, J = 8.3 Hz) |
| | 7.15 (d, 1H, J = 2.0 Hz), 7.09 (dd, 1H, J = 8.4, 2.2 Hz), |
| | 6.95 (d, 2H, J = 8.3 Hz) 6.55 (d, 1H, J = 8.4 Hz), 4.59 (s, 2H), 4.47 (dd, |
| | 1H, J = 9.7, 5.4 Hz), 4.23 (q, 2H, J = 7.1 Hz), 3.16 (m, 2H), 2.20 (s, |
| | 3H), 1.86 (s, 3H), 1.27 (t, 3H, J = 7.2 Hz) |
| Example 55 | 8.00 (d, 2H, J = 8.1 Hz), 7.67 (d, 2H, J = 8.3 Hz), 7.47 (d, 1H, J = 7.8 Hz) |
| | 7.16 (m, 2H), 7.08 (dd, 1H, J = 8.3, 2.2 Hz), 6.54 (d, 1H, J = 8.4 Hz), |
| | 4.71 (dd, 1H, J = 8.3, 7.2 Hz), 4.58 (s, 2H), 4.23 (q, 2H, |
| | J = 7.1 Hz), 3.43 (m, 2H), 2.19 (s, 3H), 1.84 (s, 3H), 1.26 (t, 3H, J = 7.2 Hz) |
| Example 56 | 7.98 (d, 2H, J = 8.1 Hz), 7.66 (d, 2H, J = 8.5 Hz), 7.13 (m, 3H), |
| | 6.81 (m, 2H), 6.54 (d, 1H, J = 8.4 Hz), 4.72 (dd, 1H, J = 9.1, 6.7 Hz), |
| | 4.59 (s, 2H), 4.23 (q, 2H, J = 7.1 Hz), 3.28 (m, 2H), 2.20 (s, |
| | 3H), 1.96 (s, 3H), 1.26 (t, 3H, J = 7.2 Hz) |
| Example 57 | 8.00 (d, 2H, J = 8.1 Hz), 7.66 (d, 2H, J = 8.3 Hz), 7.25 (d, 2H, J = 7.4 Hz), |
| | 7.18 (d, 1H, J = 1.5 Hz), 7.12 (m, 2H), 6.55 (d, 1H, J = 8.4 Hz), |
| | 4.86 (dd, 1H, J = 8.6, 6.6 Hz), 4.58 (s, 2H), 4.24 (q, 2H, J = 7.1 Hz), |
| | 3.53 (m, 2H), 2.20 (s, 3H), 1.83 (s, 3H), 1.26 (t, 3H, J = 7.1 Hz) |
| Example58 | 7.98 (d, 2H, J = 8.1 Hz), 7.66 (d, 2H, J = 8.3 Hz), 7.18 (d, 1H, J = 1.5 Hz), |
| | 7.08 (dd, 1H, J = 8.4, 2.2 Hz), 7.00 (m, 1H), 6.73 (m, 2H), |
| | 6.54 (d, 1H, J = 8.4 Hz), 4.60 (m, 3H), 4.23 (q, 2H, J = 7.1 Hz), |
| | 3.21 (m, 2H), 2.20 (s, 3H), 1.93 (s, 3H), 1.26 (t, 3H, J = 7.1 Hz) |
| Example 59 | 7.99 (d, 2H, J = 8.1 Hz), 7.67 (d, 2H, J = 8.2 Hz), 7.28 (dd, 1H, J = 8.8, |
| | 5.1 Hz), 7.18 (d, 1H, J = 1.5 Hz), 7.13 (dd, 1H, J = 8.4, 2.3 Hz), |
| | 6.87 (m, 1H), 6.56 (d, 1H, J = 8.4 Hz), 4.74 (dd, 1H, J = 9.2, 6.0 Hz), |
| | 4.59 (s, 2H), 4.23 (q, 2H, J = 7.1 Hz), 3.29 (m, 2H), 2.21 (s, |
| | 3H), 1.92 (s, 3H), 1.27 (t, 3H, J = 7.1 Hz) |
| Example 60 | 7.93 (d, 2H, J = 8.1 Hz), 7.64 (d, 2H, J = 8.0 Hz), 7.20 (d, 2H, J = 8.6 Hz), |
| | 7.14 (s, 1H), 7.08 (m, 1H), 6.76 (d, 2H, J = 8.3 Hz), |
| | 6.53 (d, 1H, J = 8.3 Hz), 4.59 (m, 1H), 4.53 (s, 2H), 4.24 (q, 2H, J = 7.1 Hz), |
| | 3.74 (s, 3H), 3.36 (d, 1H, J = 2.2 Hz), 2.19 (s, 3H), 1.89 (s, |
| | 3H), 1.28 (t, 3H, J = 7.1 Hz) |
| Example 61 | 7.60-7.76 (m, 3H), 7.11-7.17 (m, 4H), 6.90 (m, 1H), 6.55 (d, 1H, J = 8.4 Hz), |
| | 4.79 (dd, 1H, J = 8.8, 6.6 Hz), 4.59 (s, 2H), 4.23 (q, 2H, J = 7.2 Hz), |
| | 3.38 (m, 2H), 2.20 (s, 3H), 1.90 (s, 3H), 1.27 (t, 3H, J = 7.1 Hz) |
| Example 62 | 7.61-7.75 (m, 4H), 7.14 (d, 2H, J = 1.7 Hz), 7.08 (dd, 1H, J = 8.3, |
| | 2.0 Hz), 6.71 (m, 2H), 6.56 (d, 1H, J = 8.3 Hz), 4.60 (s, 2H), |
| | 4.45 (dd, 1H, J = 9.3, 5.8 Hz), 4.23 (q, 2H, J = 7.2 Hz), 3.15 (m, 2H), |
| | 2.21 (s, 3H), 1.95 (s, 3H), 1.27 (t, 3H, J = 7.1 Hz) |
| Example 63 | 7.61-7.77 (m, 3H), 7.47 (d, 1H, J = 7.5 Hz), 7.35 (m, 1H), |
| | 7.07-7.22 (m, 3H), 6.54 (d, 1H, J = 8.4 Hz), 4.70 (dd, 1H, J = 8.5, 7.0 Hz), |
| | 4.58 (s, 2H), 4.23 (q, 2H, J = 7.2 Hz), 3.43 (m, 2H), 2.19 (s, 3H), |
| | 1.84 (s, 3H), 1.27 (t, 3H, J = 7.1 Hz) |
| Example64 | 7.60-7.75 (m, 3H), 7.09-7.19 (m, 3H), 6.81 (m, 1H), 6.55 (d, 1H, J = 8.4 Hz), |
| | 4.71 (dd, 1H, J = 9.1, 6.6 Hz), 4.59 (s, 1H), 4.23 (q, 2H, J = 7.2 Hz), |
| | 3.28 (m, 2H), 2.20 (s, 3H), 1.96 (s, 3H), 1.27 (t, 3H, J = 7.1 Hz) |
| Example 65 | 7.61-7.77 (m, 3H), 7.08-7.27 (m, 5H), 6.55 (d, 1H, J = 8.4 Hz), |
| | 4.86 (dd, 1H, J = 8.7, 6.5 Hz), 4.56 (s, 1H), 4.23 (q, 2H, J = 7.2 Hz), |
| | 3.53 (m, 2H), 2.20 (s, 3H), 1.83 (s, 3H), 1.27 (t, 3H, J = 7.1 Hz) |
| Example66 | 7.75-7.61 (m, 3H), 7.15 (d, 1H, J = 1.5 Hz), 7.15 (dd, 1H, J = 8.4, |
| | 1.9 Hz), 7.00 (m, 1H), 6.79-6.69 (m, 2H), 6.55 (d, 1H, J = 8.4 Hz), |
| | 4.60 (m, 3H), 4.23 (q, 2H, J = 7.2 Hz), 3.21 (m, 2H), 2.20 (s, 3H), |
| | 1.93 (s, 3H), 1.27 (t, 3H, J = 7.1 Hz) |
| Example67 | 7.76-7.61 (m, 3H), 7.28 (m, 1H), 7.17 (d, 1H, J = 1.5 Hz), 7.12 (dd, |
| | 1H, J = 8.4, 1.9 Hz), 6.88 (m, 1H), 6.77 (dd, 1H, J = 8.9, 3.0 Hz), |
| | 6.56 (d, 1H, J = 8.4 Hz), 4.73 (dd, 1H, J = 9.2, 6.0 Hz), 4.60 (s, |
| | 1H), 4.23 (q, 2H, J = 7.2 Hz), 3.29 (m, 2H), 2.21 (s, 3H), 1.92 (s, |
| | 3H), 1.27 (t, 3H, J = 7.1 Hz) |

### Example 68

### Preparation of 2-[4-[1-[2-[4-(trifluoromethyl)phenyl]-4-methylthiazol-5-yl]-3-phenylpropylthio]-2-methylphenoxy]acetic acid of Formula X [Step G]

178 mg (0.3 mmol) of 2-[4-[1-[2-[4-(trifluoromethyl)phenyl]-4-methylthiazol-5-yl]-3-phenylpropylthio]-2-methylphenoxy]acetic acid ethyl ester was well mixed with 15 ml of ethanol. To the solution, 1.0 ml of 3 N-sodium hydroxide aqueous solution was added and the mixture was stirred at room temperature for 20 minutes. After completion of the reaction, the reaction product was adjusted to pH 2.0 with 2 N—HCl. 80% of the ethanol solvent was removed by distillation under reduced pressure, and the remaining material was extracted with aqueous salt solution and ethyl acetate. Then, the solvent was removed by distillation under reduced pressure, and the residue was purified by LH-20 column chromatography, thus obtaining 166 mg (99% yield) of the title compound.

¹H NMR (300 MHz, CDCl₃) δ7.98 (d, 2H, J=8.1 Hz), 7.68 (d, 2H, J=8.3 Hz), 7.26 (m, 3H), 7.12 (m, 3H), 6.99 (dd, 1H, J=8.4, 2.2 Hz), 6.55 (d, 1H, J=8.5 Hz), 4.64 (s, 1H), 4.20 (dd, 1H, J=8.9, 6.1 Hz), 3.85 (s, 2H), 2.73 (m, 2H), 2.38 (m, 1H), 2.20 (m, 1H), 2.17 (s, 3H), 1.89 (s, 3H).

### Example 69

### Preparation of potassium 2-[4-[2-(2-chloro-6-fluorophenyl)-1-[2-[4-(trifluoromethyl)phenyl]-4-methylthiazol-5-yl]ethylthio]-2-methylphenoxy]acetate of Formula X [Step G]

200 mg (0.31 mmol) of 2-[4-[2-(2-chloro-6-fluorophenyl)-1-[2-[4-(trifluoromethyl)phenyl]-4-methylthiazol-5-yl]ethylthio]-2-methylphenoxy]acetic acid allyl ester and 18 mg (0.015 mmol, 0.05 equivalents) of palladium tetrakistriphenylphosphine were dissolved in 10 ml of anhydrous dichloromethane and stirred at room temperature. To the reaction solution, 56 mg (0.31 mmol, 1.0 equivalent) of potassium 2-ethylhexanoate dissolved in 1 ml of anhydrous dichloromethane was slowly added. The mixture was stirred at room temperature for 1 hour, and the solvent was then removed by centrifugation. The remaining solid was washed with 10 ml of dichloromethane and 10 ml of n-hexane and dried, thus obtaining 179 mg (91% yield) of the title compound.

¹H NMR (300 MHz, D₂O) δ 7.96 (d, 2H, J=8.1 Hz), 7.66 (d, 2H, J=8.3 Hz), 7.17 (m, 5H) 6.91 (m, 1H), 6.56 (d, 1H, J=8.5 Hz), 4.79 (dd, 1H, J=8.5, 6.9 Hz), 4.66 (s, 2H), 3.39 (m, 2H), 2.18 (s, 3H), 1.87 (s, 3H).

### Examples 70 to 97

Compounds shown in Table 9 below were prepared according to the method of Example 68, and the NMR data of the prepared compounds are shown in Table 10 below. Herein, the NMR spectra of compounds wherein R_{6b} is an alkali metal (sodium or potassium) were identical to those of compounds wherein R_{6b} is hydrogen.

TABLE 9

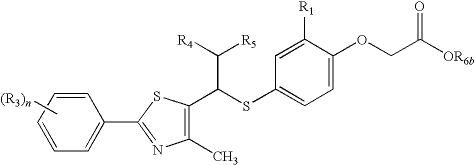

| | R₁ | 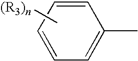 | n | R₄ | R₅ | R_{6b} |
|---|---|---|---|---|---|---|
| Example 68 | —CH₃ | 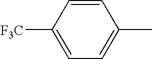 | 1 | 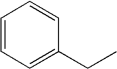 | H | H |
| Example 69 | —CH₃ | 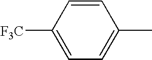 | 1 | 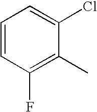 | H | K⁺ |
| Example 70 | —CH₃ | 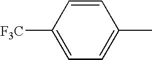 | 1 | 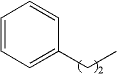 | H | H |
| Example 71 | —CH₃ | 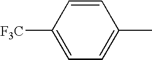 | 1 | 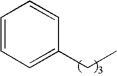 | H | H |
| Example 72 | —CH₃ | 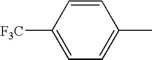 | 1 | 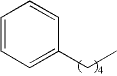 | H | H |
| Example 73 | —CH₃ | 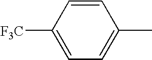 | 1 | 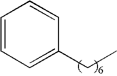 | H | H |
| Example 74 | —CH₃ | 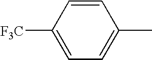 | 1 | 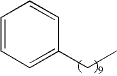 | H | H |
| Example 75 | —CH₃ | 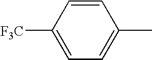 | 1 | 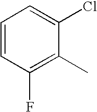 | H | H |
| Example 76 | —CH₃ | 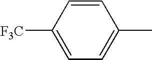 | 1 | 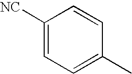 | H | H |
| Example 77 | —CH₃ | 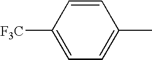 | 1 | 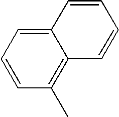 | H | H |
| Example 78 | —CH₃ | 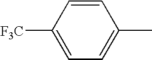 | 1 | 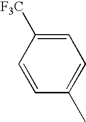 | H | H |
| Example 79 | —CH₃ | 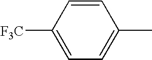 | 1 | 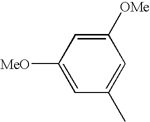 | H | H |
| Example 80 | —CH₃ | 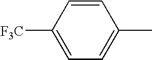 | 1 | 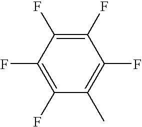 | H | H |
| Example 81 | —CH₃ | 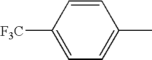 | 1 | 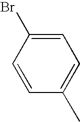 | H | H |
| Example 82 | —CH₃ | 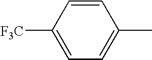 | 1 | 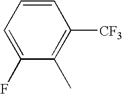 | H | H |
| Example 83 | —CH₃ | 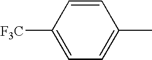 | 1 | 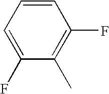 | H | H |
| Example 84 | —CH₃ | 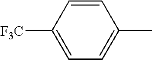 | 1 | 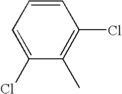 | H | H |
| Example 85 | —CH₃ | 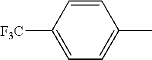 | 1 | 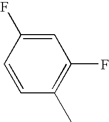 | H | H |
| Example 86 | —CH₃ | 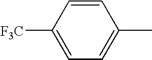 | 1 | 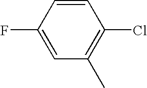 | H | H |
| Example 87 | —CH₃ | 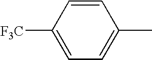 | 1 | 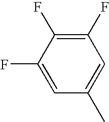 | H | H |
| Example 88 | —CH₃ | 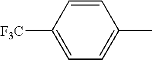 | 1 | 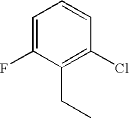 | OH | H |
| Example 89 | —CH₃ | 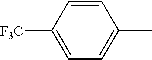 | 1 | 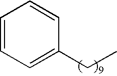 | OH | H |
| Example 90 | —CH₃ | 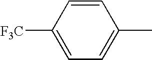 | 1 | 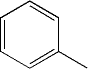 | OH | H |
| Example 91 | —CH₃ | 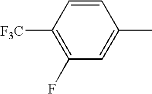 | 2 | 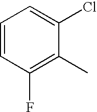 | H | H |
| Example 92 | —CH₃ | 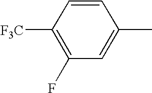 | 2 | 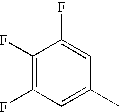 | H | H |
| Example 93 | —CH₃ | 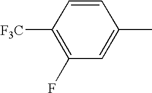 | 2 | 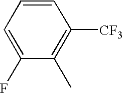 | H | H |
| Example 94 | —CH₃ | 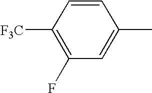 | 2 | 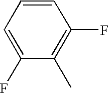 | H | H |
| Example 95 | —CH₃ | 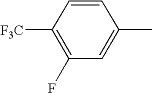 | 2 | 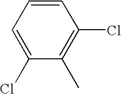 | H | H |
| Example 96 | —CH₃ | 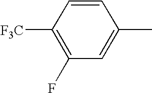 | 2 | 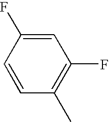 | H | H |
| Example 97 | —CH₃ | 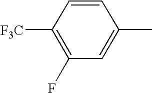 | 2 | 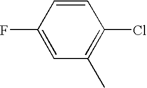 | H | H |

TABLE 10

| | ¹H NMR (300 MHz, CDCl₃) |
|---|---|
| Example68 | 7.98 (d, 2H, J = 8.1 Hz), 7.68 (d, 2H, J = 8.3 Hz), 7.26 (m, 3H), |
| | 7.12 (m, 3H), 6.99 (dd, 1H, J = 8.4, 2.2 Hz), 6.55 (d, 1H, J = 8.5 Hz), |
| | 4.64 (s, 1H), 4.20 (dd, 1H, J = 8.9, 6.1 Hz), 3.85 (s, 2H), 2.73 (m, 2H), |
| | 2.38 (m, 1H), 2.20 (m, 1H), 2.17 (s, 3H), 1.89 (s, 3H) |
| Example69 | 7.96 (d, 2H, J = 8.1 Hz), 7.66 (d, 2H, J = 8.3 Hz), 7.17 (m, 5H) |
| | 6.91 (m, 1H), 6.56 (d, 1H, J = 8.5 Hz), 4.79 (dd, 1H, J = 8.5, 6.9 Hz), |
| | 4.66 (s, 2H), 3.39 (m, 2H), 2.18 (s, 3H), 1.87 (s, 3H) |
| Example 70 | 7.94 (d, 2H, J = 8.2 Hz), 7.66 (d, 2H, J = 8.2 Hz), 7.18 (m, 6H), |
| | 6.97 (dd, 1H, J = 8.3, 1.5 Hz), 6.52 (d, 1H, J = 8.4 Hz), 5.80 (s, 1H), |
| | 4.60 (s, 2H), 4.23 (dd, 1H, J = 8.3, 6.1 Hz), 2.64 (t, 2H, J = 7.5 Hz), |
| | 2.16 (s, 3H), 2.07 (m, 1H), 1.93 (s, 3H), 1.88 (m, 1H), 1.76 (m, 2H) |
| Example 71 | 7.96 (d, 2H, J = 8.2 Hz), 7.66 (d, 2H, J = 8.2 Hz), 7.20 (m, 6H), |
| | 6.99 (dd, 1H, J = 8.3, 1.5 Hz), 6.53 (d, 1H, J = 8.4 Hz), 5.39 (s, 1H), |
| | 4.61 (s, 2H), 4.23 (dd, 1H, J = 8.8, 6.1 Hz), 2.59 (m, 2H), 2.17 (s, 3H), |
| | 1.88 (m, 1H), 1.94 (s, 3H), 1.91 (m, 1H), 1.65 (m, 2H), 1.46 (m, 2H) |
| Example72 | 7.96 (d, 2H, J = 8.4 Hz), 7.66 (d, 2H, J = 8.5 Hz), 7.63 (s, 1H), |
| | 7.20 (m, 6H), 6.98 (dd, 1H, J = 8.3, 2.2 Hz), 6.54 (d, 1H, J = 8.5 Hz), |
| | 4.59 (s, 2H), 4.22 (dd, 1H, J = 9.0, 6.0 Hz), 2.58 (t, 2H, J = 7.6 Hz), |
| | 2.17 (s, 3H), 2.03 (m, 1H), 1.93 (s, 3H), 1.89 (m, 1H), 1.61 (m, 2H), |
| | 1.42 (m, 4H) |
| Example 73 | 7.96 (d, 2H, J = 8.1 Hz), 7.71 (s, 1H), 7.66 (d, 2H, J = 8.3 Hz), |
| | 7.20 (m, 6H), 6.98 (dd, 1H, J = 8.4, 2.2 Hz), 6.54 (d, 1H, J = 8.5 Hz), |
| | 4.62 (s, 2H), 4.23 (dd, 1H, J = 9.0, 6.0 Hz), 2.58 (t, 2H, J = 7.6 Hz), |
| | 2.17 (s, 3H), 2.03 (m, 1H), 1.95 (s, 3H), 1.89 (m, 1H), 1.61 (m, 2H), |
| | 1.28 (m, 8H) |
| Example 74 | 7.96 (d, 2H, J = 8.1 Hz), 7.66 (d, 2H, J = 8.3 Hz), 7.18 (m, 6H), |
| | 6.99 (dd, 1H, J = 8.3, 1.8 Hz), 6.54 (d, 1H, J = 8.5 Hz), 4.91 (s, 1H), |
| | 4.61 (s, 2H), 4.24 (dd, 1H, J = 9.0, 5.9 Hz), 2.58 (t, 2H, J = 7.6 Hz), |
| | 2.17 (s, 3H), 2.03 (m, 1H), 1.95 (s, 3H), 1.85 (m, 1H), 1.59 (m, 2H), |
| | 1.28 (m, 14H) |
| Example 75 | 7.96 (d, 2H, J = 8.1 Hz), 7.66 (d, 2H, J = 8.3 Hz), 7.17 (m, 5H) |
| | 6.91 (m, 1H), 6.56 (d, 1H, J = 8.5 Hz), 4.79 (dd, 1H, J = 8.5, 6.9 Hz), |
| | 4.66 (s, 2H), 3.39 (m, 2H), 2.18 (s, 3H), 1.87 (s, 3H) |
| Example76 | 7.96 (d, 2H, J = 8.1 Hz), 7.67 (d, 2H, J = 8.2 Hz), 7.53 (d, 2H, J = 8.2 Hz), |
| | 7.21 (d, 2H, J = 8.2 Hz), 7.16 (d, 2H, J = 1.5 Hz), 7.05 (dd, |
| | 1H, J = 10.3, 1.8 Hz), 6.57 (d, 1H, J = 8.5 Hz), 4.65 (s, 2H), 4.50 (dd, |
| | 1H, J = 9.4, 5.7 Hz), 3.30 (m, 3H), 2.19 (s, 3H), 1.81 (m, 1H) |
| Example 77 | 7.95 (d, 2H, J = 8.1 Hz), 7.74 (m, 1H), 7.67 (d, 2H, J = 8.2 Hz), |
| | 7.72 (d, 2H, J = 8.5 Hz), 7.66 (d, 2H, J = 8.3 Hz), 7.56 (s, 1H), 7.42 (m, |
| | 2H), 7.18 (m, 2H), 7.05 (dd, 1H, J = 8.2 2.2 Hz), 6.55 (d, 1H, J = 8.5 Hz), |
| | 4.61 (m, 4H), 3.40 (m, 2H), 2.17 (s, 3H), 1.78 (m, 1H) |
| Example78 | 7.96 (d, 2H, J = 8.1 Hz), 7.67 (d, 2H, J = 8.2 Hz), 7.49 (d, 2H, J = 8.0 Hz), |
| | 7.21 (d, 2H, J = 8.0 Hz), 7.16 (d, 2H, J = 1.5 Hz), 7.05 (dd, |
| | 1H, J = 10.3, 1.9 Hz), 6.56 (d, 1H, J = 8.5 Hz), 5.34 (s, 1H), 4.63 (s, |
| | 2H), 4.52 (dd, 1H, J = 9.4, 5.7 Hz), 3.30 (m, 2H), 2.18 (s, 3H), |
| | 1.79 (m, 1H) |
| Example79 | 7.95 (d, 2H, J = 8.1 Hz), 7.66 (d, 2H, J = 8.3 Hz), 7.16 (d, 1H, J = 1.6 Hz), |
| | 7.07 (m, 2H), 6.55 (d, 1H, J = 8.5 Hz), 6.29 (t, 1H, J = 2.2 Hz) |
| | 6.24, (d, 2H, J = 2.2 Hz), 4.62 (s, 2H), 4.53 (dd, 1H, J = 9.5, 5.5 Hz), |
| | 3.70 (s, 6H), 3.16 (m, 2H), 2.18 (s, 3H), 1.84 (s, 3H) |
| Example80 | 8.58 (s, 1H), 7.96 (d, 2H, J = 8.1 Hz), 7.67 (d, 2H, J = 8.3 Hz), |
| | 7.14 (d, 1H, J = 1.7 Hz), 7.07 (dd, 1H, J = 8.4, 2.2 Hz), 6.57 (d, 1H, J = 8.5 Hz), |
| | 4.66 (m, 3H), 3.31 (m, 2H), 2.19 (s, 3H), 2.01 (s, 3H) |
| Example81 | 7.95 (d, 2H, J = 8.1 Hz), 7.66 (d, 2H, J = 8.3 Hz), 7.34 (m, 2H) |
| | 7.15 (d, 1H, J = 1.6 Hz), 7.05 (dd, 1H, J = 8.4, 2.2 Hz), 6.95 (m, 2H), |
| | 6.77 (s, 1H), 6.55 (d, 1H, J = 8.5 Hz), 4.63 (s, 2H), 4.47 (dd, 1H, J = 9.6, |
| | 5.6 Hz), 3.18 (m, 2H), 2.18 (s, 3H), 1.80 (s, 3H) |
| Example82 | 8.44 (s, 1H), 7.96 (d, 2H, J = 8.1 Hz), 7.66 (d, 2H, J = 8.3 Hz), |
| | 7.47 (d, 1H, J = 7.8 Hz) 7.34 (m, 1H), 7.19 (t, 1H, J = 9.1 Hz), 7.13 (d, 1H, |
| | J = 1.7 Hz), 7.05 (dd, 1H, J = 8.4, 2.2 Hz), 6.54 (d, 1H, J = 8.4 Hz), |
| | 4.70 (dd, 1H, J = 8.0, 8.0 Hz), 4.61 (s, 2H), 3.43 (m, 2H), 2.17 (s, |
| | 3H), 1.80 (s, 3H) |
| Example83 | 7.95 (d, 2H, J = 8.1 Hz), 7.65 (d, 2H, J = 8.4 Hz), 7.13 (m, 3H), |
| | 6.81 (m, 2H), 6.55 (d, 1H, J = 8.4 Hz), 6.01 (s, 1H), 4.71 (dd, 1H, J = 8.9, |
| | 6.8 Hz), 4.63 (s, 2H), 3.30 (m, 2H), 2.18 (s, 3H), 1.91 (s, 3H) |
| Example84 | 7.97 (d, 2H, J = 8.1 Hz), 7.66 (d, 2H, J = 8.3 Hz), 7.27 (d, 2H, J = 8.2 Hz), |
| | 7.18 (d, 1H, J = 1.6 Hz), 7.11 (m, 2H), 6.55 (m, 2H), |
| | 4.85 (dd, 1H, J = 8.4, 6.8 Hz), 4.62 (s, 2H), 3.53 (m, 2H), 2.18 (s, 3H), |
| | 1.80 (s, 3H) |
| Example85 | 7.95 (d, 2H, J = 8.1 Hz), 7.66 (d, 2H, J = 8.3 Hz), 7.38 (s, 1H), |
| | 7.16 (d, 1H, J = 1.6 Hz), 7.03 (m, 2H), 7.00 (m, 1H), 6.74 (m, 2H), 6.56 (d, |
| | 1H, J = 8.5 Hz), 4.59 (m, 3H), 3.17 (m, 2H), 2.18 (s, 3H), 1.87 (s, 3H) |
| Example86 | 7.96 (d, 2H, J = 8.1 Hz), 7.82 (s, 1H), 7.66 (d, 2H, J = 8.3 Hz), |
| | 7.28 (dd, 1H, J = 8.8, 5.1 Hz), 7.18 (d, 1H, J = 1.5 Hz), 7.08 (dd, 1H, J = 8.4, |
| | 2.2 Hz), 6.87 (m, 1H), 6.80 (dd, 1H, J = 8.9, 3.0 Hz), 6.56 (d, 1H, |
| | J = 8.5 Hz), 4.73 (dd, 1H, J = 9.0, 6.2 Hz), 4.63 (s, 2H), 3.29 (m, 2H), |
| | 2.18 (s, 3H), 1.87 (s, 3H) |
| Example87 | 7.98 (d, 2H, J = 8.1 Hz), 7.67 (d, 2H, J = 8.2 Hz), 7.15 (s, 1H), |
| | 7.09 (dd, 1H, J = 8.4, 2.2 Hz), 6.73 (t, 1H, J = 7.6 Hz) 6.56 (d, 1H, J = 8.4 Hz), |
| | 4.60 (s, 2H), 4.46 (dd, 1H, J = 9.3, 5.8 Hz), 3.69 (s, 6H), |
| | 3.15 (m, 2H), 2.21 (s, 3H), 1.95 (s, 3H), |
| Example 88 | 7.91 (d, 2H, J = 8.1 Hz), 7.61 (d, 2H, J = 8.0 Hz), 7.10 (m, 5H), |
| | 6.42 (d, 1H, J = 8.1 Hz), 5.32 (br s, 1H), 4.45 (m, 1H), 4.33 (s, 2H), |
| | 4.23 (m, 1H), 3.12 (m, 1H), 2.95 (br s, 1H), 2.89 (m, 1H), 2.09 (s, 3H), |
| | 2.04 (s, 3H) |
| Example 89 | 8.01 (d, 2H, J = 8.2 Hz), 7.67 (d, 2H, J = 8.1 Hz), 7.20 (m, 7H), |
| | 6.62 (d, 1H, J = 8.5 Hz), 4.67 (s, 2H), 4.43 (d, 1H, J = 2.9 Hz), 3.81 (m, |
| | 1H), 3.78 (br s, 1H), 2.57 (t, 2H, J = 7.7 Hz), 2.23 (s, 3H), 2.19 (s, |
| | 3H), 1.58 (m, 2H), 1.42-1.21 (m, 14H) |
| Example 90 | 8.00 (d, 2H, J = 8.2 Hz), 7.67 (d, 2H, J = 8.1 Hz), 7.26-7.17 (m, 7H), |
| | 6.73 (d, 2H, J = 8.5 Hz), 5.12 (d, 1H, J = 3.3 Hz), 4.67 (s, 2H), |
| | 4.63 (d, 1H, J = 3.4 Hz), 4.15 (br s, 1H), 2.24 (s, 3H), 1.80 (s, 3H) |
| Example 91 | 7.60-7.73 (m, 3H), 7.08-7.17 (m, 5H), 6.90 (m, 1H), 6.57 (d, 1H, J = 8.3 Hz), |
| | 4.78 (dd, 1H, J = 8.7, 6.7 Hz), 4.63 (s, 2H), 3.38 (m, 2H), |
| | 2.18 (s, 3H), 1.87 (s, 3H) |
| Example 92 | 7.61-7.72 (m, 3H), 7.20 (bs, 1H), 7.14 (d, 1H, J = 2.0 Hz), 7.04 (dd, |
| | 1H, J = 8.4, 2.2 Hz), 6.73 (m, 1H), 6.57 (d, 1H, J = 8.3 Hz), 4.64 (s, |
| | 2H), 4.45 (dd, 1H, J = 9.2, 5.9 Hz), 3.16 (m, 2H), 2.19 (s, 3H), |
| | 1.90 (s, 3H) |
| Example 93 | 7.60-7.74 (m, 3H), 7.48 (d, 1H, J = 7.8 Hz), 7.35 (m, 1H), 7.19 (t, 1H, |
| | J = 9.1 Hz), 7.13 (d, 1H, J = 1.6 Hz), 7.06 (dd, 1H, J = 8.4, 2.2 Hz), |
| | 6.86 (bs, 1H), 6.55 (d, 1H, J = 8.5 Hz), 4.70 (dd, 1H, J = 8.2, 7.3 Hz), |
| | 4.62 (s, 2H), 3.43 (m, 2H), 2.17 (s, 3H), 1.80 (s, 3H) |
| Example 94 | 7.61-7.74 (m, 4H), 7.08-7.21 (m, 3H), 6.83 (m, 1H), 6.58 (d, 1H, J = 8.5 Hz), |
| | 4.72 (dd, 1H, J = 9.0, 6.7 Hz), 4.65 (s, 2H), 3.31 (m, 2H), |
| | 2.20 (s, 3H), 1.94 (s, 3H) |
| Example 95 | 7.60-7.74 (m, 3H), 7.24-7.27 (m, 2H), 7.18 (d, 1H, J = 1.6 Hz), |
| | 7.08-7.13 (m, 2H), 7.00 (bs, 1H), 6.56 (d, 1H, J = 8.5 Hz), 4.85 (dd, |
| | 1H, J = 8.6, 6.7 Hz), 4.62 (s, 2H), 3.53 (m, 2H), 2.18 (s, 3H), 1.80 (s, |
| | 3H) |
| Example 96 | 7.60-7.72 (m, 3H), 7.15 (m, 2H), 6.98-7.08 (m, 2H), 6.70-6.79 (m, 2H), |
| | 6.56 (d, 1H, J = 8.5 Hz), 4.60-4.63 (m, 3H), 3.21 (m, 2H), 2.18 (s, |
| | 3H), 1.89 (s, 3H) |
| Example 97 | 7.60-7.73 (m, 3H), 7.55 (bs, 1H), 7.28 (m, 1H), 7.17 (d, 1H, J = 1.7 Hz), |
| | 7.09 (dd, 1H, J = 8.4, 2.2 Hz), 6.84-6.91 (m, 1H), 6.79 (dd, 1H, J = 8.9, |
| | 3.0 Hz), 6.57 (d, 1H, J = 8.5 Hz), 4.73 (dd, 1H, J = 9.0, 6.2 Hz), |
| | 4.63 (s, 2H), 3.29 (m, 2H), 2.18 (s, 3H), 1.88 (s, 3H) |

### Test Example 1

### Activity and Cytotoxicity Tests

The compounds prepared in Examples were tested for PPARδ activity by a transfection assay. In addition, the compounds were tested for selectivity for PPARα and PPARγ, the subtypes of PPARs, and also tested for toxicity by a MTT assay.

Transfection Assay

A transfection assay was performed using CV-1 cells. The culture of the cells was performed using a DMEM medium (10% FBS, DBS (delipidated) and 1% penicillin/streptomycin) on a 96-well plate in a 5% carbon dioxide-containing incubator at 37° C. The test was performed in four steps consisting of cell inoculation, transfection, treatment of the compounds, and analysis of results. Specifically, CV-1 cells were inoculated onto a 96-well plate at a concentration of 5,000 cells/well, and after 24 hours, the cells were transfected. In the cell transfection, full-length PPARs plasmid DNA, reporter DNA that has luciferase activity and thus allows the identification of PPARs, and β-galactosidase DNA that provides the information of transfection efficiency, were used as transfection reagents. Each of the compounds developed in the present invention was dissolved in dimethylsulfoxide (DMSO) and transfected into the cells at various concentrations using media. The cells were cultured in an incubator for 24 and then lysed with lysis buffer. The lysed cells were measured for luciferase and β-galactosidase activities using a luminometer and a microplate reader. The measured luciferase values were normalized with the β-galactosidase values and graphed. From the graph, EC₅₀ values were determined.

The EC₅₀ values of the compounds prepared in Examples 47-97 according to the present invention were mostly less than 50 nM, and the compounds showed at least 10,000-fold selectivity for PPARα and PPARγ.

MTT Assay

The compounds of Examples 47-97 according to the present invention were tested for cytotoxicity using a MTT assay. MTT is a water-soluble yellow substance, but if it is introduced into living cells, it will be degenerated into a water-insoluble purple crystal due to dehydrogenase contained in mitochondria. If this substance is dissolved in dimethylsulfoxide and then measured for absorbance at 550 nm, cytotoxicity can be assayed. The test method is as follows.

CV-1 cells were first inoculated onto a 96-well plate at a concentration of 5,000 cells/well. The inoculated cells were cultured in a 5% carbon dioxide-containing humidified incubator at 37° C. for 24 hours, and then treated with the inventive compounds at various concentrations. After 24 hours of culture, a MTT reagent was added to the cultured cells. After 15 minutes of culture, the resulting purple crystal was dissolved in dimethylsulfoxide and then measured for absorbance using a microplate reader. From the measured absorbance, cytotoxicity was assayed.

The test results showed that most of the inventive compounds had no cytotoxicity even at a concentration of 90 μM.

| | | | | LC₅₀ |
|---|---|---|---|---|
| | EC₅₀ PPARδ | EC₅₀ PPARα | EC₅₀ PPARγ | Cytotoxicity |
| Sample | (nM) | (nM) | (nM) | (μM) |
| Example 47 | 6.5 | ia | ia | >100 |
| Example 68 | 2.7 | ia | ia | >100 |
| Example 70 | 6.1 | ia | ia | 91 |
| Example 71 | 38.8 | ia | ia | 93 |
| Example 72 | 11.5 | ia | ia | 92 |
| Example 73 | 57.2 | ia | ia | 94 |
| Example 74 | 132.1 | ia | ia | >100,000 |
| Example 75 | 2.1 | ia | ia | >100 |
| Example 76 | 22.1 | ia | ia | >100 |
| Example 77 | 75.0 | ia | ia | >100 |
| Example 78 | 13.3 | ia | ia | >100 |
| Example 79 | 46.4 | ia | ia | >100 |
| Example 80 | 46.4 | ia | ND | >100 |
| Example 81 | 11.5 | ND | ia | 92 |
| Example 82 | 2.6 | ia | ND | >100 |
| Example 83 | 2.6 | >100,000 | >10,000 | >100 |
| Example 84 | 2.1 | ia | ia | >100 |
| Example 85 | 3.4 | ia | ia | >100 |
| Example 86 | 8.7 | ia | ia | >100 |
| Example 87 | 5.3 | ia | ia | >100 |
| Example 91 | 3.9 | ia | ia | >10 |
| Example 92 | 21.6 | ia | ia | >30 |
| Example 93 | 2.6 | ia | ia | >10 |
| Example 94 | 0.9 | ia | ia | >10 |
| Example 95 | 2.3 | ia | ia | >10 |
| Example 96 | 4.8 | >10,000 | ia | >10 |
| Example 97 | 17.4 | ia | ia | >30 |

### INDUSTRIAL APPLICABILITY

The present invention provides novel thiazole derivatives as peroxisome proliferator-activated receptor δ (PPARδ)-activating ligands, which can be used for treatment of obesity, hyperlipidemia, arteriosclerosis and diabetes, as well as their intermediates and preparation methods thereof. The present invention is useful to provide novel thiazole derivative compounds as PPARδ-activating ligands.

What is claimed is:

1. Thiazole derivatives of Formula I as racemates or optical isomers: 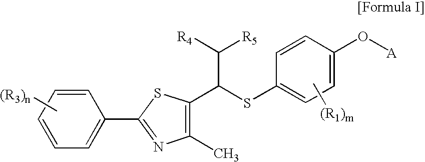
wherein A is hydrogen, R₂ or 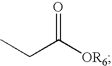
R₁ is a hydrogen atom, a C₁₋₄ alkyl group, a C₁₋₄ alkyloxy group, a C₁₋₄ alkylthioxy group, a C₁₋₄ alkylamine group, a fluorine atom or a chlorine atom;
M is an integer from 0 to 4;
R₂ is a phenol-protecting group selected from among C₁₋₄ lower alkyl groups, allyl groups, alkylsilyl groups, alkylarylsilyl groups and a tetrahydropyranyl group;
R₃ groups are different from each other and denote a hydrogen atom, a halogen atom, or a C₁₋₄ alkyl or alkoxy group substituted or unsubstituted with halogen;
N is an integer from 0 to 5;
R₄ is 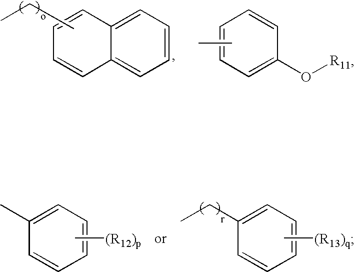
R₅ is a hydrogen atom, a hydroxyl group or a C₁₋₄ alkyl group;
R₆ is a carboxylic acid protecting group having C₁₋₄ alkyl, an allyl group, a hydrogen atom or an alkali metal;
R₁₁ is an arylaminoalkyl group or an alkylaminoalkyl group;
R₁₂ is a halogen atom, a cyano group, or a C₁₋₄ alkyl or alkoxy group substituted with unsubstituted with halogen;
R₁₃ is a hydrogen atom, a halogen atom, a cyano group, a C₁₋₄ alkyl or alkoxy group substituted with unsubstituted with halogen;
o, p and q are each independently an integer from 1 to 5; and
r is an integer from 1 to 9.

2. The thiazole derivatives of claim 1, which are represented by Formula VI: 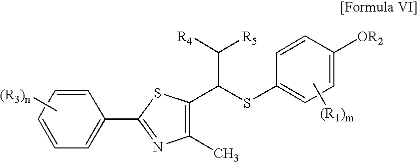
wherein R₁ to R₅, m and n have the same meanings as defined in Formula I.

3. The thiazole derivatives of claim 1, which are represented by Formula VII: 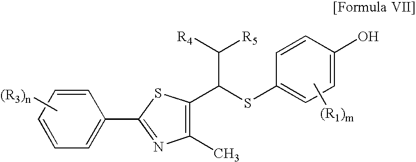
wherein R₁, R₃ to R₅, m and n have the same meanings as defined in Formula I.

4. The thiazole derivatives of claim 1, which are represented by Formula IX: 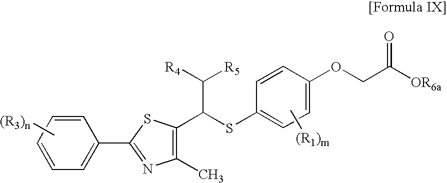
wherein R₁, R₃ to R₅, m and n have the same meanings as defined in Formula I, and R₆ₐ is a carboxylic acid protecting group having C₁₋₄ alkyl or an allyl group.

5. The thiazole derivatives of claim 1, which are represented by Formula X: 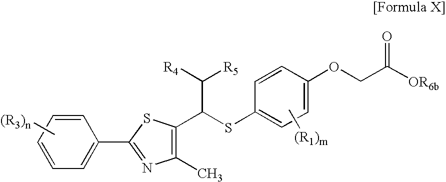
wherein R₁, R₃ to R₅, m and n have the same meanings as defined in Formula I, and R_{6b} is a hydrogen atom or an alkali metal.

6. A method for preparing thiazole derivatives, comprising the steps of:
a) reacting a 4-halogen phenol compound of Formula II with a phenol-protecting alkylsilyl group in the presence of a base to prepare a compound of Formula III;
b) subjecting the compound of Formula III to halogen-to-lithium substitution, and then to reaction with sulfur and a compound of Formula IV so as to prepare a compound of Formula V; and
c) reacting the compound of Formula V with a strong base and an electrophilic compound so as to prepare a compound of Formula VI: 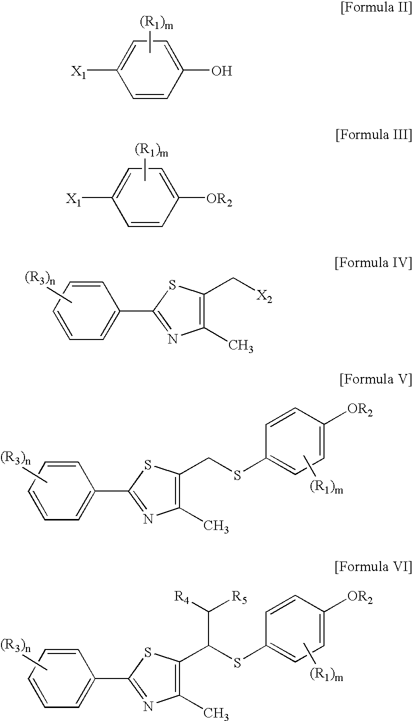
wherein X₁ denotes a bromine atom or an iodine atom, X₂ denotes a chlorine atom, a bromine atom, an iodine atom, or a leaving group having high reactivity in nucleophilic substitution reaction, and the remainder has the same meanings as defined in Formula I.

7. The method of claim 6, which further comprises the step of: d) removing the phenol-protecting silyl group of a compound of Formula VI so as to prepare a compound of Formula VII: 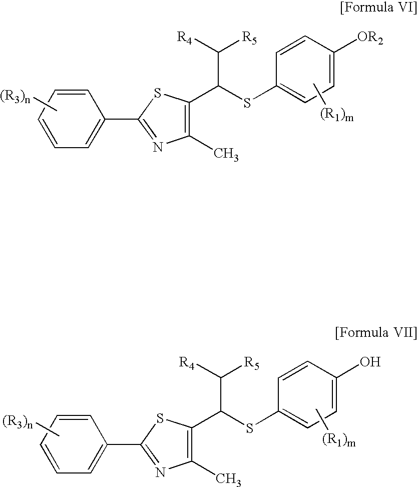
wherein R₁ to R₅, m and n have the same meanings as defined in Formula I.

8. A method for preparing thiazole derivatives, comprising the steps of:
reacting a 4-halogen phenol compound of Formula II with a Grignard reagent, subjecting the reaction product to halogen-to-lithium substitution and then reacting the reaction product with sulfur and the compound of Formula IV so as to form a thioether compound; and
reacting the thioether compound with a strong base without separation and then with an electrophilic compound so as to prepare a compound of Formula VII: 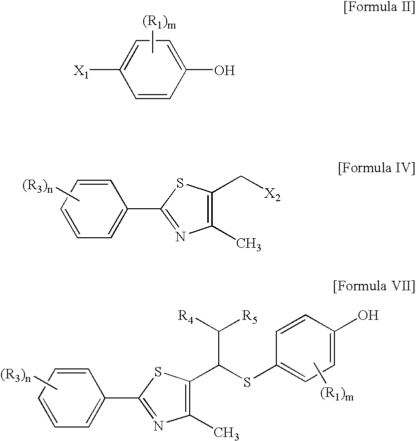
wherein X₁ denotes a bromine atom or an iodine atom, X₂ denotes a chlorine atom, a bromine atom, an iodine atom, or a leaving group having high reactivity in electrophilic substitution reaction, and R₁, R₃ to R₅, m and n have the same meanings as defined in Formula I.

9. The method of claim 7 or 8, which further comprises the step of:
e) reacting the compound of Formula VII with an alkyl halogen acetate of Formula VIII in the presence of an inorganic salt so as to prepare a compound of Formula IX: 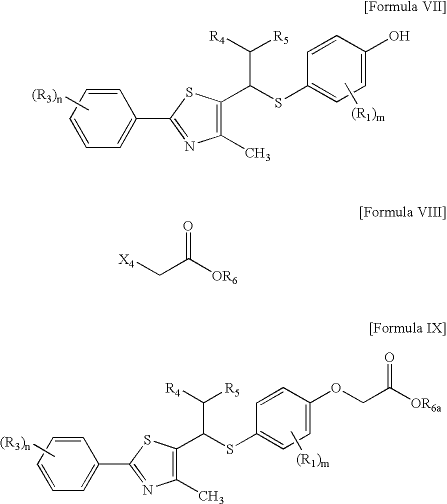
wherein R₁, R₃ to R₅, m and n have the same meanings as defined in Formula I, and R₆ₐ is a carboxylic acid protecting group having C₁₋₄ alkyl or an allyl group, and X₄ is a chlorine atom, a bromine atom or an iodine atom.

10. The method of claim 9, which further comprises the step of:
subjecting the compound of Formula IX to carboxylic acid ester hydrolysis with a water-soluble inorganic salt in an alcohol solution so as to prepare a compound of Formula X: 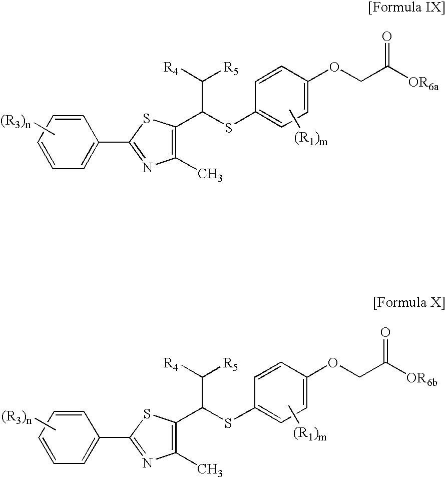
wherein R₁, R₃ to R₅, m and n have the same meanings as defined in Formula I, R₆ₐ is a carboxylic acid protecting group having C₁₋₄ or an allyl group, and R_{6b} is a hydrogen atom or an alkali metal.

11. The method of claim 9, which further comprises the step of:
subjecting the compound of Formula IX to allyl ester-to-salt substitution using a metal salt in an organic solvent in the presence of a palladium tetrakistriphenylphosphine catalyst so as to prepare a compound of Formula X: 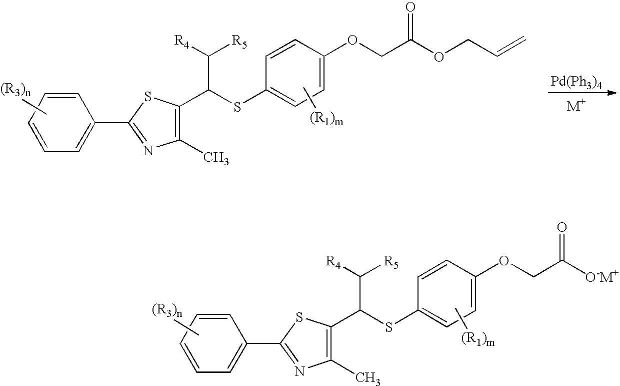
wherein R₁, R₃ to R₅, m and n have the same meanings as defined in Formula I, and M is an alkali metal.

12. An agent for treating diabetes, comprising as an active ingredient a thiazole derivative represented by Formula I.

13. An agent for preventing and treating obesity, comprising as an active ingredient a thiazole derivative represented by Formula I.

14. An agent for preventing and treating arteriosclerosis, comprising as an active ingredient a thiazole derivative represented by Formula I.

15. An agent for preventing and treating hyperlipidemia, comprising as an active ingredient a thiazole derivative represented by Formula I.

16. Health food supplement, health beverage, food additive, functional cosmetic and animal feed compositions comprising as an active ingredient a thiazole derivative represented by Formula I.

17. A composition for activating a peroxisome proliferator-activated receptor δ (PPARδ), comprising as an active ingredient a thiazole derivative represented by Formula I.
